(19) United States
(12) Patent Application Publication
(10) Pub. No.: US 2012/0294886 A1
(43) Pub. Date: Nov. 22, 2012

(54) PEPTIDE CONJUGATES FOR SUPPRESSING AN IMMUNE RESPONSE, METHODS OF MAKING AND USES THEREFOR

(75) Inventors: Judith Greer, Mt. Glorious (AU); Elisabeth Trifilieff, Strasbourg (FR)

(73) Assignee: The University of Queensland, St. Lucia (AU)

(21) Appl. No.: 13/109,863

(22) Filed: May 17, 2011

(51) Int. Cl. A61K 39/00; C07K 4/00; A61P 37/08; C12N 5/078; A61P 37/06; C07K 2/00; C07K 1/113

(52) U.S. Cl. 424/194.1; 530/300; 530/345; 435/375

(57) ABSTRACT

This application relates to peptide conjugates comprising peptides that suppress or otherwise inhibit an unwanted or undesirable immune response attached to lipid moieties, and methods for suppressing immune responses, including preventing, inhibiting, treating or decreasing unwanted or undesirable immune responses including autoimmune or allergic immune responses using these peptide conjugates.

### RELATED APPLICATIONS

This application is a national stage application under 35 U.S.C. §1.111(a) of PCT/AU2009/001501, filed Nov. 18, 2009, published as WO 2010/057251 A1 and published on May 27, 2010, which claims priority to U.S. Provisional Patent Application No. 61/115,881, filed Nov. 18, 2008 and U.S. Provisional Patent Application No. 61/224,429, filed Jul. 9, 2009, which applications and publications are incorporated herein by reference and made a part hereof in their entirety, and the benefit of priority of which is claimed herein.

### FIELD OF THE INVENTION

This invention relates generally to compounds and methods for suppressing immune responses, including preventing, inhibiting, treating or decreasing unwanted or undesirable immune responses including autoimmune or allergic immune responses.

### BACKGROUND OF THE INVENTION

The body may raise unwanted or undesirable immune responses in a number of situations, including autoimmune responses, transplant rejections and allergic immune responses.

### Autoimmune Responses

Lymphocyte development occurs during foetal development, and to a lesser extent throughout the remainder of life. Part of this development includes immunological tolerance; the process by which T and B lymphocytes (T and B cells) of the immune system, which recognise self antigens, are deleted before they develop into fully immunocompetent cells.

Autoimmune responses occur when the body fails to recognise a self antigen, resulting in an immune response against the body's own cells and tissues. Any disease that results from such an immune response to a self antigen is termed an autoimmune disease.

Autoimmune diseases affect approximately 5% of the global population. Rheumatoid arthritis (RA) affects more than 5.2 million individuals and multiple sclerosis (MS) affects approximately 2.5 million individuals globally.

There is currently no cure for any autoimmune disease. Most current therapies only aim to improve the signs and symptoms or treat the complications brought about by the disease. While disease-modified drugs represent advances for management of some diseases including RA and MS, none are fully effective as they help minimise symptoms and improve the quality of life for patients but do not change the course of the diseases. Side-effects (albeit of varying nature and severity) are common when taking these drugs. Additionally, the long-term use of these disease-modifying drugs has been associated with some serious side-effects such as liver damage, kidney damage, thyroid functional problems, depression, and posing a risk to pregnancy.

### Transplant Rejection

Immunocompetent T cells will raise an immune response to foreign antigens including those within transplants containing nucleated cells. Matching the Major Histocompatibility Complex (MHC) type of the donor and recipient increases the success rate of grafts, but perfect matching is possible only when donor and recipient are related and even in these cases genetic differences at other loci still trigger rejection.

Therefore almost all grafts require long-term immunosuppression. Currently available immunosuppressive therapies are generally non-specific and therefore inhibit all immune responses, both undesirable and desirable. Such therapies can cause significant toxicity and increase the risk of cancer and infection.

Demand for transplant therapy is on the rise, causing waiting lists for suitable donors to rise also.

### Allergic Immune Reactions

An allergic immune response occurs where the immune system reacts to a normally innocuous environmental antigen. The immune response results from interaction between the antigen and the antibody or T cells produced by earlier exposure to the same antigen.

Allergic immune responses include type I (or immediate) hypersensitivity immune responses which are antibody (typically IgE) mediated, and type IV (delayed type) hypersensitivity immune responses which are cell mediated and antibody independent. IgE-mediated allergies include hayfever, skin inflammation (urticaria), food allergies, asthma and systemic anaphylaxis. Cell mediated allergic diseases include contact dermatitis, tuberculin reaction, and chronic transplant rejection.

The prevalence of allergic immune diseases has increased in many parts of the world over the past 20 to 30 years. It is estimated that approximately 11% of the US population and 5.5% of the UK population suffer from allergic rhinitis, while 3% of the US population and 9.4% of the UK population suffer from asthma.

Existing drugs to treat allergic immune reactions help to alleviate the symptoms of allergy, and are imperative in the recovery of acute anaphylaxis, but play little role in chronic treatment of allergic diseases Immunotherapy, such as densensitisation or hyposensitisation or the use of monoclonal antibodies has proven effective in treating some allergic immune responses, but are not suitable for all allergic immune diseases.

### Using Peptides to Ameliorate Aberrant Immune Responses

Antigens can be categorised as either class I or class II peptides depending on whether they attach to MHC class I molecules or MHC class II molecules. MHC class I molecules usually bind short peptides of 8-10 residues. Typical class I antigens are those derived from cytosolic pathogens. The length of peptides bound by MHC class II molecules is not constrained. Typical class II antigens are intravesicular pathogens, extracellular pathogens and extracellular toxins.

The two classes of MHC molecules are recognised by different functional classes of T cells, leading to the release of different sets of effector molecules. MHC class I molecules are recognised by cytotoxic T cells which kill the cell bearing the antigen. MHC class II molecules are recognised by T helper cells which either activate effector molecules such as cytokines or activate B cells to secrete immunoglobulins. In this way, an immune response appropriate for the antigen is raised.

Peptides which comprise sequences that prevent, inhibit, treat or decrease an unwanted or undesirable immune response are generally known. It is believed that many such peptides are based on known antigens but have modified sequences, normally at sites where T cell receptors or MHC anchor positions interact. These peptides can be classified as class I or class II peptides depending on which class of MHC molecule they interact with, although the mechanism of their interaction is not well understood yet. There is much interest in therapies based on these peptides as they will probably be disease-specific and therefore not associated with the side effects of some current immunosuppressive therapies.

It has been shown in experimental models that such peptides can be used to ameliorate an aberrant immune response mediated through T helper cells and therefore have application in the prevention or treatment of autoimmune diseases and allergic disorders, and in improving transplant acceptance.

In these models, antigen presenting cells are typically pulsed or otherwise contacted with an immunosuppressive peptide. It is believed that the peptide attaches to appropriate MHC molecules on the surface of the cells and suppresses an unwanted or undesirable T helper cell-mediated immune response. This is in contrast to the way that an immune response is raised to an antigen, where the antigen is processed internally by an antigen presenting cell (APC), including attachment to an MHC molecule, transportation to the cell surface, and presentation of the MHC-antigen complex to T helper cells. This difference may explain why these models have not yielded effective immunosuppression. Such pulsing methods therefore have questionable effectiveness in clinical applications. Further, in 2000 two multicenter phase II clinical trials of immunosuppressive peptides for multiple sclerosis (MS) were halted early after severe immediate-type hypersensitivity reactions in patients with MS, which were due in large measure to the large amounts of peptide injected into patients in an attempt to overcome the poor immunogenicity of the synthetic peptide vaccines (Bielekova, et al., 2000 and Kappos et al., 2000).

In recognising some of the problems associated with the methods that use naked peptides, the inventors of U.S. Pat. No. 6,737,057 designed a molecule consisting of an immunoglobulin (or portion thereof) linked with a peptide that is a T cell receptor antagonist. The immunoglobulin enables the molecule to be transported into an APC for presentation down the class II pathway Immunoglobulins are expensive, time-consuming and cumbersome to produce. Further, there is a risk that the patient will raise an immune response to the immunoglobulin if it is foreign.

Other methods of internalising an immunogenic peptide have been previously described. Cell penetrating peptides (CPPs) are described in Stewart et al., 2008. However, being peptides themselves, it is not known what effect a CPP would have if linked to an immunosuppressive peptide. Carrier proteins such as bovine serum albumin (BSA) and keyhole limpet hemocyanin have also been used to transport immunogenic peptides. Such proteins are not likely to be suitable carrier molecules for immunosuppressive peptides because, as for immunoglobulins, there is a risk that the patient will raise an immune reaction to the carrier protein. In fact, commercially available BSA is often cationised so that it can provide an enhanced immunogenic response.

WO 97/49425 describes a vaccine comprising an antigen and a carrier molecule that are linked through a labile bond. The application states that the site of attachment of the carrier molecule to the peptide affects the immunogenicity of the antigen. The inventors state that an S site of attachment yields a more immunogenic product than an N site of attachment. However, both were found to elicit class II immune responses, with the inventors noting that the N-attached carrier molecule induced a high antibody response.

In work leading up to the present invention, the present inventors surprisingly discovered that immunosuppressive peptides with lipid moieties attached at an S-site on the peptide are processed by the class II pathway for improved class II presentation, while immunosuppressive peptides with lipid moieties attached at an N-site on the peptide tend to form micelles and are processed through the class I pathway for improved class I presentation. Based on this discovery, the present inventors consider that better prophylactic and therapeutic immunosuppressive responses can be elicited through the class II pathway by attaching immunosuppressive peptides that are processed and presented through MHC class II molecules to a membrane permeating lipid moiety through a thioester linkage, and that better prophylactic and therapeutic immunosuppressive responses can be achieved through the class I pathway by attaching immunosuppressive peptides that are processed and presented through MHC class I molecules to a membrane permeating lipid molecule through an amide linkage.

The above discoveries have been reduced to practice in novel compounds and methods for promoting immunosuppressive responses.

### SUMMARY OF THE INVENTION

Accordingly, in one aspect, the present invention provides a peptide conjugate for suppressing or otherwise inhibiting an MHC class II response, the peptide conjugate comprising a peptide comprising an amino acid sequence that suppresses or otherwise inhibits an unwanted or undesirable immune response and that is processed and presented by an MHC class II molecule, and a lipid moiety which is attached to the peptide through a thioester linkage, or a pharmaceutically acceptable salt thereof.

In another aspect, the present invention provides a peptide conjugate for suppressing or otherwise inhibiting an MHC class I response, the peptide conjugate comprising a peptide comprising an amino acid sequence that suppresses or otherwise inhibits an unwanted or undesirable immune response and that is processed and presented by an MHC class I molecule, and a lipid moiety which is attached to the peptide through an amide linkage, or a pharmaceutically acceptable salt thereof.

In a further aspect, the present invention provides a composition comprising the peptide conjugate of the present invention.

In yet a further aspect, the present invention provides a composition consisting essentially of the peptide conjugate of the present invention.

In yet a further aspect, the present invention provides a composition consisting of the peptide conjugate of the present invention.

In another aspect, the present invention provides a composition comprising a peptide conjugate of the present invention but excluding a separate antigen that corresponds to the sequence of the peptide, which antigen elicits the unwanted or undesirable immune response.

According to another aspect of the present invention, there is provided a method of making a peptide conjugate according to the first-mentioned aspect, the method comprising linking a peptide comprising an amino acid sequence that suppresses or otherwise inhibits an unwanted or undesirable immune response and that is processed and presented by an MHC class II molecule to a lipid moiety through a thioester linkage.

According to another aspect of the present invention, there is provided a method of making a peptide conjugate according to the second-mentioned aspect, the method comprising linking a peptide comprising an amino acid sequence that suppresses or otherwise inhibits an unwanted or undesirable immune response and that is processed and presented by an MHC class I molecule to a lipid moiety through an amide linkage.

In yet another aspect, the present invention provides a method for suppressing or otherwise inhibiting an unwanted or undesirable immune response in a subject, the method comprising administering to the subject the peptide conjugate or composition of the invention.

In some embodiments, the subject has or is afflicted with an autoimmune, an allergic immune or an allograft immune response.

In some embodiments, the method comprises identifying that the subject has an autoimmune, an allergic immune or an allograft immune response.

Another aspect of the present invention relates to a method for suppressing or otherwise inhibiting an unwanted or undesirable immune response to a target antigen in a subject, the method comprising administering to the subject the peptide conjugate or composition of the invention, wherein the sequence of the peptide corresponds to the sequence of the target antigen.

In some embodiments, the subject has or is afflicted with an autoimmune, an allergic immune or an allograft immune response.

In some embodiments, the method comprises identifying that the subject has an autoimmune, an allergic immune or an allograft immune response.

A further aspect of the present invention provides a method for preventing, inhibiting, treating or decreasing an autoimmune, an allergic immune or an allograft immune response in a subject, the method comprising administering to the subject the peptide conjugate or composition of the invention.

In some embodiments, the present invention provides a method for treating an autoimmune, an allergic immune or an allograft immune response in a subject, the method comprising administering to the subject the peptide conjugate or composition of the invention.

In some embodiments, the subject has or is afflicted with an autoimmune, an allergic immune or an allograft immune response.

In some embodiments, the method comprises identifying that the subject has an autoimmune, an allergic immune or an allograft immune response.

Yet further aspect of the present invention provides a method for preventing, inhibiting, treating or decreasing an autoimmune, an allergic immune or an allograft immune response to a target antigen in a subject, the method comprising administering to the subject the peptide conjugate or composition of the invention, wherein the sequence of the peptide corresponds to the sequence of the target antigen.

In some embodiments, the present invention provides a method of treating an autoimmune, an allergic immune or an allograft immune response to a target antigen in a subject, the method comprising administering to the subject the peptide conjugate or composition of the invention, wherein the sequence of the peptide corresponds to the sequence of the target antigen.

In some embodiments, the subject has or is afflicted with an autoimmune, an allergic immune or an allograft immune response.

In some embodiments, the method comprises identifying that the subject has an autoimmune, an allergic immune or an allograft immune response.

In another aspect of the present invention, there is provided a use of a peptide conjugate or a composition of the invention in the manufacture of a medicament for suppressing or otherwise inhibiting an unwanted or undesirable immune response in a subject, including an unwanted or undesirable immune response to a target antigen.

In another aspect of the present invention, there is provided a use of a peptide conjugate or a composition of the invention in the manufacture of a medicament for preventing, inhibiting, treating or decreasing an autoimmune, an allergic immune or an allograft immune response in a subject, including an autoimmune, an allergic immune or an allograft immune response to a target antigen in a subject.

In some embodiments, the present invention provides a use of a peptide conjugate or a composition of the invention in the manufacture of a medicament for treating an autoimmune, an allergic immune or an allograft immune response in a subject, including an autoimmune, an allergic immune or an allograft immune response to a target antigen in a subject.

In yet another aspect, the present invention provides a method for producing an immunosuppressive antigen presenting cell, the method comprising contacting an antigen presenting cell or antigen presenting cell precursor with the peptide conjugate or composition of the invention for a time and under conditions sufficient for the peptide or a processed form thereof to be presented by the antigen presenting cell or antigen presenting cell precursor.

### BRIEF DESCRIPTION OF THE DRAWINGS

FIG. 1 is a graphical representation of some of the results in Example 2. Mice were immunised with the peptide shown above each graph, and then lymph node cells extracted therefrom were tested for their proliferative responses to a nonacylated (circle), S-palm (square), or N-palm (triangle) forms of the same peptide. Each point on the graph represents the SI (mean±SD of three to five repetitions of each experiment) at a particular peptide concentration.

FIG. 2 shows some of the results in Example 3, where the uptake of different peptides by the macrophages was examined. Uptake of the peptides into the macrophages was monitored by flow cytometry (at 15 min) and by confocal microscopy after 1-, 5-, 15-, and 30-min incubation. The bar in this Figure represents 5 μm. The S-palmitoyalted and N-palmitoyalted peptides were taken up much more rapidly and to much higher concentrations into macrophages than were non-palmitoylated peptides, as indicated by the stronger staining of the palmitoylated peptides—the palmitoylated peptides could easily be visualised inside the cells after 1 min, whereas the non-palmitoylated peptides could barely be visualised, even after 30 min incubation. This is also seen in the flow cytometric plot at 15 min, where the fluorescence intensity of staining with the palmitoylated peptides is several logs higher than the non-palmitoylated peptide.

FIG. 3 shows some results of Example 3, demonstrating that there were differences in the route of uptake of S-palm or N-palm peptides. S-palm peptide colocalised rapidly and strongly with endosomes. N-palm peptide did not colocalise strongly with endosomes in most cells. Further, S-palm peptide colocalised strongly with lysosomes. N-palm peptide did not colocalise strongly with lysosomes in most cells, and there was no time-dependent increase of this localisation. The bars in the graphs in FIG. 3 represent the percentage colocalisation (mean±SE) in at least 100 cells. The term “nd” means not done.

FIG. 4 shows some results of Example 3 where the colocalisation of N-palm peptide and S-palm peptide to endoplasmic reticulum (ER) were compared. The results show percentage colocalisation after incubation with biotinylated peptide for 30 or 60 minutes and staining to detect ER or peptide. Colocalisation of N-palm peptide and ER increased after 30 minutes of incubation, compared with colocalisation of S-palm peptide with ER.

FIG. 5 shows some results of Example 3 where S-palm peptide colocalised strongly with MHC class II, but only a small percentage colocalised with MHC class I (p<0.0001). In contrast, N-palm peptide colocalised strongly with MHC class I and to a much lesser degree with MHC class II (p<0.0001).

FIG. 6 shows the results of Example 7. Mice that had developed EAE were injected with PBS (control), the APL A188, or the S-palmitoylated APL (S-palm-A188). The figure shows that the control group continued to develop more severe EAE, whereas the mean score of the S-palm-A188-treated group did not increase above the score that the mice were at when they were injected with the treatment. The response of the mice treated with A188 was intermediate between the control group and the S-palm-A188-treated group.

FIG. 7 shows the results of the flow cytometric analysis of Example 8. LNC from mice immunised with either A188 or S-palm A188 were analysed. The analysis confirmed that the S-palm A188 induces an increase in the number of regulatory cells. FIG. 7 shows the percentages of regulatory T cells in LNC from mice immunized with either A188 or S-palm A188. The baseline level of regulatory T cells (i.e. no antigen group) is increased in the S-palm A188-treated mice, and those levels are increased further upon stimulation of the LNC with either A188 or PLP178-191. These results may explain in part the increased immunomodulatory capacity of the S-palm A188.

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

The term “about” is used herein to refer to conditions (e.g., amounts, concentrations, time etc) that vary by as much as 30%, preferably by as much as 20%, and more preferably by as much as 10%, 9%, 8%, 7%, 6%, 5%, 4%, 3%, 2% or 1% to a specified condition.

The term “allograft” as used herein refers to a graft containing cells, tissues, organisms etc that are of different genetic constitution to the recipient.

The term “anergy” as used herein refers to a suppressed response, or a state of non-responsiveness, to a specified antigen or group of antigens by an immune system. For example, T lymphocytes and B lymphocytes are anergic when they cannot respond to their specific antigen under optimal conditions of stimulation.

By “antigen” is meant all, or part of, a protein, peptide, or other molecule or macromolecule capable of eliciting an immune response in a vertebrate animal, especially a mammal. Such antigens are also reactive with antibodies from animals immunised with that protein, peptide, or other molecule or macromolecule.

Throughout this specification, unless the context requires otherwise, the words “comprise,” “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements. By “consisting of” is meant including, and limited to, whatever follows the phrase “consisting of”. Thus, the phrase “consisting of” indicates that the listed elements are required or manadatory, and that no other elements may be present.

By “corresponds to” or “corresponding to” is meant a peptide which encodes an amino acid sequence that displays substantial similarity to an amino acid sequence in a target antigen. In general the peptide will display at least about 30, 40, 50, 55, 60, 65, 70, 75, 80, 85, 90, 91, 92, 93, 94, 95, 96, 97, 98, 99% similarity to at least a portion of the target antigen.

By “effective amount,” in the context of preventing, inhibiting, treating or decreasing an immune response, is meant the administration of that amount of peptide compound to an individual in need thereof, either in a single dose or as part of a series, that is effective for achieving that prevention, inhibition, treatment or decrease. The effective amount will vary depending upon the health and physical condition of the individual, the taxonomic group of individual, the formulation of the composition, the assessment of the medical situation, and other relevant factors. It is expected that the amount will fall in a relatively broad range that can be determined through routine trials.

The term “immunosuppressive peptide” refers to a peptide that inhibits an immune response to an antigen or that causes the immune system to become unresponsive to an antigen. This includes peptides that cause insensitivity of T cells to T cell receptor-mediated stimulation. Representative “immunosuppressive peptides” may induce tolerance to an antigen, or stimulate suppressor cell (e.g., regulatory T cell) function, or induce anergy in or clonal deletion of T cells in an antigen-specific manner.

By “isolated” is meant material that is substantially or essentially free from components that normally accompany it in its native state.

The term “membrane permeating lipid moiety” refers to a moiety that is able to penetrate and pass through a membrane. The membrane permeating moiety is able to transport the peptide to which it is conjugated through a membrane.

The terms “patient,” “subject,” “host” or “individual” used interchangeably herein, refer to any subject, particularly a vertebrate subject, and even more particularly a mammalian subject, for whom prophylaxis or therapy is desired. Suitable vertebrate animals that fall within the scope of the present invention include, but are not restricted to, any member of the subphylum Chordata including primates, rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc), marine mammals (e.g., dolphins, whales), reptiles (snakes, frogs, lizards etc), and fish. A preferred subject is a human suffering from an autoimmune disease, an allergic immune disease or who is an allograft recipient. However, it will be understood that the aforementioned terms do not imply that symptoms are present.

By “pharmaceutically acceptable excipient or diluent” is meant a solid or liquid filler, diluent or encapsulating substance that may be safely used in topical or systemic administration.

The term “pharmaceutically compatible salt” as used herein refers to a salt which is toxicologically safe for human and animal administration. This salt may be selected from a group including hydrochlorides, hydrobromides, hydroiodides, sulfates, bisulfates, nitrates, citrates, tartrates, bitartrates, phosphates, malates, maleates, napsylates, fumarates, succinates, acetates, terephthalates, pamoates and pectinates. For example, pharmaceutically acceptable salts include the acid addition salts (formed with free amino groups of the peptide) and which are formed with inorganic acids such as, for example, hydrochloric or phosphoric acids, or organic acids such as acetic, oxalic, tartaric, maleic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

“Peptide” refers to a polymer of amino acid residues and to variants and synthetic analogues of the same. Thus, this term encompasses amino acid polymers in which all of the amino acid residues are naturally occurring and amino acid polymers in which one or more amino acid residues is a synthetic non-naturally occurring amino acid, such as a chemical analogue of a corresponding naturally occurring amino acid, as well as to naturally-occurring amino acid polymers.

The term “peptide conjugate” refers to a compound that includes at least two moieties, a peptide moiety and a lipid moiety, that are joined or linked together by a covalent bond.

By “prevention”, “prevent”, “prevented”, and the like is meant to include prophylactic treatment, including but not limited to (1) preventing, inhibiting, or delaying the onset of, or the development of an autoimmune, an allergic immune or an allograft immune response, or (2) preventing, inhibiting, or delaying a symptom of the immune response.

By “recombinant peptide” is meant a peptide made using recombinant techniques, i.e., through the expression of a recombinant polynucleotide.

By “treatment,” “treat,” “treated” and the like is meant to include therapeutic treatment, including but not limited to (1) relieving, altering, reversing, affecting, inhibiting the development of, inhibiting the progression of, ameliorating, or curing an autoimmune, an allergic immune or an allograft immune response, (2) relieving, altering, reversing, affecting, inhibiting the development of, inhibiting the progression of, ameliorating, or curing a symptom of the immune response, or (3) reducing the number of, or lengthening the time between relapses of the immune response, or reducing the symptoms of a relapse.

### 2. Peptides

The present invention contemplates peptide conjugates that comprise any peptide comprising an amino acid sequence that suppresses or otherwise inhibits an unwanted or undesirable immune response. Many such peptides are known.

For example, altered peptide ligands (APLs) are one class of such peptides. Interaction of T cell receptors with APLs has been shown to skew the cytokine profile of responding T cells, or to induce anergy. Therefore, it has been shown in experimental models that APLs can be used to stimulate or enhance a tolerogenic immune response mediated through T-cells and also have application in the prevention, inhibition, treatment or decrease of autoimmune, allergic immune or allograft immune responses.

APLs are based on known antigens but have modified sequences, normally at sites where T cell receptors or MHC anchor positions interact with the antigen on which the APL is based. The sequence modification in the APL is usually determined by using a panel of peptides based on the known antigen, where at least one residue in each peptide is a conservative or non-conservative substitution of the residue in the known antigen. Each peptide is then tested for its ability to bind the MHC molecule of interest and to induce responses in T cells specific for the native antigen.

In some embodiments APLs are based on antigens that are processed and presented through MHC class I molecules.

In some embodiments APLs are based on antigens that are processed and presented through MHC class II molecules.

In some embodiments APLs are derived from antigen sequences where the MHC anchoring amino acids are determined, for example by alanine scanning. The MHC anchoring amino acids may then be substituted with another amino acid or may be deleted from the sequence. If more than one amino acid is involved as an MHC anchor, more than one of these amino acids may be substituted or deleted.

In some embodiments, APLs are derived from CNS myelin proteins, such as proteolipid protein (PLP), myelin basic protein (MBP) and myelin oligodendrocyte glycoprotein (MOG). Such APLs may be useful in stimulating or enhancing a tolerogenic immune response to autoimmune neurodegenerative diseases such as multiple sclerosis.

Many known APLs specific to particular diseases or conditions are described in the art. Illustrative examples of these are listed in the tables below.

TABLE 1
Known APLs processed and presented through class I molecules

| | | | Amino acid seq of | | |
|---|---|---|---|---|---|
| Immune response | Antigen | Species | original antigen | Amino acid change | Reference |
| Allograft | EBV | Human, HLA- | FLRGRAYGL (SEQ | A → F | Ely et al. |
| | | B*0801 | ID NO: 13) | | |
| | HA-1 | Human, HLA-A2 | VLHDDLLEA (SEQ | position 3 (3G and 3S) | den Haan et al. |
| | (minor histocompatibility | | ID NO: 14) | | |
| | antigen) | | | | |
| Diabetes | insulin B15-23 peptide | Mouse, H-2Kd | LYLVCGERG (SEQ | 6 G → H | de Marquesini |
| | | | ID NO: 15) | | et al. |
| | | | LYLVCGERG (SEQ | 8 R → L | |
| | | | ID NO: 15) | | |
| | influenza virus | Mouse, H-2Kd | 512-520 | 517 A → G | Hartemann- |
| | hemagglutinin (HA) | | | | Heurtier et al. |

TABLE 2
Known APLs processed and presented through class II molecules

| | | | Amino acid seq of | | |
|---|---|---|---|---|---|
| Immune response | Antigen | Species | original antigen | Amino acid change | Reference |
| Allergic | Bos d 2¹⁶ | Humans, DR4 | 127-142 | 135 N → D | Kinnunen T et al. |
| | (a lipocalin allergen from | | | | |
| | cow dander) | | | | |
| Allograft | Dby mH | Mouse, H-2Ek | | 490 R → H | Chen T et al., |
| | | | | | 2004 |
| Arthritis—rheumatoid | type II collagen | Human, HLA- | 263-272 | Three APLs: | Li R et al. |
| | | DR4 | (FKGEQGPKGE) | FKGEAGPAGE | |
| | | | (SEQ ID NO: 16) | (SEQ ID NO: 17) | |
| | | | | FKAEAGPAGE | |
| | | | | (SEQ ID NO: 18) | |
| | | | | FKGEAGPAAE | |
| | | | | (SEQ ID NO: 19) | |
| | HC gp-39 | Human, HLA- | 263-275 | 265 F → | Boots A et al. |
| | (cartilage glycoprotein-39) | DR4 | | diphenylalanine | |
| | type II collagen | Mouse, I-Aq | 245-270 | 260 → A | Myers L et al. |
| | | | | 261 → | |
| | | | | hydroxyproline | |
| | | | | 263 → N | |
| | type II collagen | Human, HLA- | 256-271 | 262 G → A | Ohnishi Y et al. |
| | | DRBT0101 | | | |
| | type II collagen | Mouse, I-Aq | 256-271 | 262 G → A | Wakamatsu et al. |
| Arthritis—experimental | Hsp60 | Rat, RT1.BI | 180-188 | 183 L → A | Prakken B et al. |
| (adjuvant induced) | | | | | |
| Diabetes—type 1 | insulin B-chain [B(9-23)] | Mouse (also | 9-23 | 16 Y → A | Alleva D et al. |
| | | human) | | 19 C → A | |
| | human glutamic acid | Human, DRB1 | 555-567 | 561 I → M | Gebe J et al. |
| | decarboxylase 65 | | | | |
| | (hGAD65) epitope | | | | |
| Myasthenia gravis | nicotinic acetylcholine | Mouse | dual analog peptide, | 262 → K | Katz-Levy et al. |
| | receptor (AChR) | | 259-271: 195-212 | 207 → A | |
| Psoriasis | keratin 17 | HLA-DRB1*07 | | Two APLs: | Shen Z et al. |
| | | | 118-132 | 119 R → A | |
| | | | 348-362 | 355 L → A | |
| Guillain-Barré syndrome | bovine P2 | Rat, RT1.BL | 60-70 | shortened (amino | Offenhäusser M |
| | | | | acids 62-69 only) | et al. |
| Sjögren's syndrome | M3R AA216-230 | | VPPGECFIQFLSEPT | 223 I → K | Naito et al. |
| | | | (SEQ ID NO: 20) | | |
| | | | VPPGECFIQFLSEPT | 224 Q → A | Naito et al. |
| | | | (SEQ ID NO: 20) | | |
| | | | | Akpvvhlfanivtprtp | |
| Multiple sclerosis | NBI 5788 | Humans | MBP 83-99 | (SEQ ID NO: 21) | Crowe et al. |
| EAE | myelin basic protein | Mouse | MBP 87-99 | 91 K → A | Karin et al. |
| | proteolipid protein | mouse | PLP139-151 | 144 W → L; 147 H | Kuchroo et al. |
| | proteolipid protein | mouse | PLP139-151 | → R | Nicholson et al |
| | proteolipid protein | mouse | PLP178-191 | 144 W → Q | Greer et al., |
| | | | | 188 F → A | 1997 |

In some embodiments where the peptide conjugate suppresses or otherwise inhibits an MHC class II response, the peptide that comprises a sequence that suppresses or otherwise inhibits an unwanted or undesirable immune response is a peptide, such as an APL, that comprises a sequence that includes at least one amino acid residue having a free thiol group that is capable of forming a thioester with the lipid moiety.

In some embodiments the peptide contains at least one cysteine residue in its sequence. In some embodiments the peptide comprises one amino acid residue having a free thiol group in its sequence The free thiol group may be present in the side chain of an amino acid residue in the peptide. An amino acid residue having a free thiol group in its side chain may be a natural or common amino acid such as L-cysteine, or a non-natural or uncommon amino acid such as D-cysteine, L-homocysteine, D-homocysteine, L-penicillamine or D-penicillamine.

In other embodiments where the peptide conjugate suppresses or otherwise inhibits an MHC class II response, the peptide that comprises a sequence that suppresses or otherwise inhibits an unwanted or undesirable immune response is a peptide that does not normally include an amino acid residue having a free thiol group, but for the purposes of this invention has a further amino acid residue, in addition to the sequence, at the C-terminus or N-terminus of the peptide, having a free thiol group. In some embodiments, the further amino acid residue is a cysteine, homocysteine or penicillamine residue, especially a cysteine residue.

The peptide of the peptide conjugate that suppresses or otherwise inhibits an MHC class II response may also be capped with an N-terminal capping group and/or a C-terminal capping group. These groups may provide biostability to the peptide or may provide a free thiol group which may be acylated. For example, the C-terminus may be capped with an amino group or substituted amino group to form an amidated C-terminus, including —CONH₂, —CONH(alkyl), —CON(alkyl)₂, —CONH—(CH₂)₁₋₃SH wherein the —CO group is derived from the C-terminal carboxyl group and the term “alkyl” in each use independently refers to a saturated hydrocarbon group having 1 to 6 carbon atoms, such as methyl, ethyl, propyl, isopropyl, butyl, 2-methylpropyl, tert-butyl, pentyl and hexyl. Suitable N-terminal capping groups include acyl groups including —C(O)(CH₂)₁₋₃SH. If the N- or C-terminal capping group includes a thiol group, this group may be acylated in accordance with the invention.

N-terminal and C-terminal capping groups may be introduced into the peptide by acylation or amidation as known in the art. Thiol containing N-terminal capping groups or C-terminal capping groups such as 3-mercaptopropionic acid or 2-mercaptoethylamine are commercially available.

In some embodiments where the peptide conjugate suppresses or otherwise inhibits an MHC class I response, the peptide that comprises a sequence that suppresses or otherwise inhibits an unwanted or undesirable immune response is a peptide, such as an APL, that comprises a sequence that includes at least one amino acid residue having a free amino group that is capable of forming an amide with the lipid moiety. The free amino group may be the amino group of the N-terminal amino acid or a free amino group on an amino acid side chain such as a lysine side chain.

The peptide of the peptide conjugate that suppresses or otherwise inhibits an MHC class I response may also be capped with a C-terminal capping group and/or, when the N-terminal amino group is not part of the amide linkage with the lipid moiety of the conjugate, an N-terminal capping group. These groups may provide biostability to the peptide. For example, the C-terminus may be capped with an amino group or substituted amino group to form an amidated C-terminus, including—CONH₂, —CONH(alkyl), —CON(alkyl)₂, where the —CO group is derived from the C-terminal carboxyl group and the term “alkyl” in each use independently refers to a saturated hydrocarbon group having 1 to 6 carbon atoms, such as methyl, ethyl, propyl, isopropyl, butyl, 2-methylpropyl, tert-butyl, pentyl and hexyl. Suitable N-terminal capping groups include acyl groups including —C(O)(CH₂)₁₋₃SH. If the N- or C-terminal capping group includes an amino group, this group may be acylated in accordance with the invention.

N-terminal and C-terminal capping groups may be introduced into the peptide by acylation or amidation as known in the art. Thiol containing N-terminal capping groups or C-terminal capping groups such as 3-mercaptopropionic acid or 2-mercaptoethylamine are commercially available.

The peptides of the peptide conjugate of this invention may be of any suitable size that can be utilised to suppress or inhibit unwanted or undesirable immune response. A number of factors can influence the choice of peptide size. In some embodiments, the peptide has 6 to 60 amino acid residues, especially 10 to 50, 11 to 40, 12 to 30, 12 to 25 or 12 to 20 amino acid residues, more especially 12 to 20, 12 to 19, 12 to 18, 12 to 17, 12 to 16 or 12 to 15 amino acid residues.

The size of a peptide can be chosen such that it includes, or corresponds to the size of T cell epitopes and their processing requirements. Practitioners in the art will recognise that class I-restricted T cell epitopes usually range between 8-10 amino acid residues in length, and that class II-restricted T cell epitopes usually range between 12 and 25 amino acid residues in length. The epitopes may or may not require natural flanking residues. Another important feature of class II-restricted epitopes is that they generally contain a ‘core section’ of 9-10 amino acid residues in the middle of the sequence which bind specifically to class II MHC molecules, and with flanking sequences either side of this ‘core section’ that stabilise binding by associating with conserved structures on either side of class II MHC molecules in a sequence independent manner. Thus the functional region of class II-restricted epitopes is typically less than about 15 amino acid residues long. From the foregoing, it is advantageous, but not essential, that the size of the peptide is at least 6, 7, 8, 9, 10, 11, 12, 13, 14, 15, 20, 25, 30 amino acid residues. Suitably, the size of the peptide is no more than about 60, 50, 40, 30 amino acid residues. In certain advantageous embodiments, the size of the peptide is sufficient for presentation by an MHC class I molecule of an antigen presenting cell. In certain other advantageous embodiments, the size of the peptide is sufficient for presentation by an MHC class II molecule of an antigen presenting cell.

### 3. Lipid Moieties

The present invention stems at least in part from the determination that the immunosuppressive effect of a peptide that comprises a sequence that suppresses or otherwise inhibits an unwanted or undesirable immune response may be improved by attaching the peptide to a membrane permeating lipid moiety. The lipid moiety in the peptide conjugates of the present invention is therefore any suitable lipid moiety that allows the peptide to permeate or pass through membranes. In specific embodiments the lipid moiety permeates membranes of antigen-presenting cells such as dendritic cells and macrophages.

Many suitable lipid moieties are known in the art.

Suitably, the lipid moiety comprises a saturated or monounsaturated fatty acid having 8 to 18 carbon atoms, especially 10 to 18 carbon atoms, 12 to 16 carbon atoms or 14 to 16 carbon atoms, more especially 14 to 16 carbon atoms. In certain embodiments the fatty acid is a saturated fatty acid. Exemplary fatty acids include n-dodecanoic acid (lauric acid), n-tetradecanoic acid (myristic acid), n-hexadecanoic acid (palmitic acid), n-octadecanoic acid (stearic acid), hexadec-9-enoic acid (palmitoleic acid) and octadec-9-enoic acid (oleic acid).

In particular embodiments, the fatty acid lipid moiety is selected from a myristoyl group or a palmitoyl group.

### 4. Peptide Conjugates

In the present application, the peptide conjugates comprise a peptide comprising a sequence that suppresses or otherwise inhibits an unwanted or undesirable immune response and a lipid moiety. The peptide and the lipid moiety may be linked by a covalent thioester linkage, or by an amide linkage.

The thioester linkage is formed between a free thiol group on the peptide and a carboxylic acid on the lipid moiety. The amide linkage is formed between an amine group on the peptide and a carboxylic acid on the lipid moiety.

In some embodiments, the peptide conjugate comprises one lipid moiety. In other embodiments, the peptide conjugate comprises more than one lipid moiety linked to the peptide through thioester or amide linkages. The limit on the number and nature of lipid moieties attached to a peptide is the ability of the peptide conjugate to pass through a membrane without becoming anchored in the membrane. In particular embodiments, the peptide conjugate comprises one or two lipid moieties.

### 5. Methods of Making Peptides and Peptide Conjugates

The peptides of the peptide conjugates of the present invention may be prepared or obtained by methods known in the art including solution phase or solid phase synthesis (Jones, Amino Acid and Peptide Synthesis, Oxford Chemistry Primers, 1992), isolation from natural sources or preparation by recombinant methodology (Sambrook et al. Molecular Cloning: A laboratory manual, 2^{nd} Edition, Cold Spring Harbour Laboratory Press Plain view, N.Y., 1989).

In some embodiments, the peptides include at least one, especially one, thiol-containing residue, especially cysteine, in the peptide sequence. In some embodiments, the at least one thiol-containing residue is incorporated in the sequence during solution phase, solid phase or recombinant synthesis or is present in the isolated naturally occurring peptide. In some embodiments, the at least one thiol-containing residue is incorporated in the sequence at the N-terminus or C-terminus of the peptide during solution phase, solid phase or recombinant synthesis or after isolation of a naturally occurring peptide.

In some embodiments, the peptides include at least one residue having an amino-containing side chain, such as lysine. In some embodiments, the at least one residue having an amino-containing side chain is incorporated in the sequence during solution phase, solid phase or recombinant synthesis or is present in the isolated naturally occurring peptide.

In some embodiments, the free amino group is the N-terminal amino group and the peptide is synthesised under normal solid phase, solution phase or recombinant synthesis or the naturally occurring peptide is isolated and if required, the N-terminal amino group deprotected of any protecting group that may have been introduced during synthesis or isolation.

In some embodiments, the peptides are synthesised using solid phase synthesis techniques using known strategies such as t-butoxycarbonyl (BOC) or 9-fluorenylmethoxycarbonyl (Fmoc), N-protection and deprotection strategies and carboxylic acid activation as known in the art. Suitable side chain protection and deprotection strategies to use will depend on whether selective deprotection of side chain functional groups is required during or after synthesis. Suitable side chain protecting groups are known in the art, for example, in Greene & Wuts, Protective Groups in Organic Synthesis, 3^{rd} Edition, 1999, John Wiley & Sons.

In some embodiments, the peptides are synthesised using Fmoc solid phase synthesis and activation using coupling agents such as N—N′-carbonyldiimidazole (CDI), N,N′-dicyclohexylcarbodiimide (DCC), HBTU, benzotriazole-1-yl-oxy-tris-(dimethylamino)-phosphonium hexafluorophosphate (BOP), 3-(Diethoxy-phosphoryloxy)-3H-benzo[d][1,2,3]-triazin-4-one (DEPBT), N,N′-diisopropylcarbodiimide (DIC), 1-ethyl-3-(3-dimethylaminopropyl)carbodiimide hydrochloride (EDC HCl), 2-(1H-2-Azabenzotriazol-1-yl)-1,1,3,3-tetramethyluronium hexafluorophosphate methanaminium (HATU), 1-hydroxy-7-azabenzotriazole (HOAt), N-hydroxybenzotriazole (HOBT), hydroxy-3,4-dihydro-4-oxo-1,2,3-benzotriazine (HOOBT), 1H-benzotriazolium-1-[bis(dimethylamino)methylene]-5-chloro-hexafluorophosphate-3-oxide (HCTU), 6-chloro-1-hydroxybenzotriazole (Cl-HOBO, benzotriazol-1-yl-oxytripyrrolidinophosphonium hexafluorophosphate (PyBOP), bromo-tris-pyrrolidinophosphonium hexafluorophosphate (PyBrOP), O-(benzotriazol-1-yl)-N,N,N′,N′-tetramethyluronium tetrafluoroborate (TBTU), N,N,N′,N′-tetramethyl-O-(3,4-dihydro-4-oxo-benzotriazin-3-yOuronium tetrafluoroborate (TDBTU), 2-(7-aza-1H-benzotriazol-1-yl)-1,1,3,3-tetramethyluronium tetrafluoroborate (TATU), O—(N-succinimidyl)-1,1,3,3-tetramethyluronium tetrafluoroborate (TSTU) and 4,5-dicyanoimidazole, especially BOP.

During synthesis of the peptides that contain at least one free thiol group, the at least one thiol-containing residue is protected with a protecting group that may be selectively removed in the presence of other side chain or N-terminal protecting groups. Suitable thiol protecting groups are described in Greene & Wuts, ibid, and include, for example, methoxytrityl (Mmt) which is labile in 2% trifluoracetic acid (TFA) and t-butyl sulfenyl (StBu) which is labile in tributylphosphine.

In some embodiments, the peptide is synthesised using solid phase synthesis on a resin and the at least one thiol-containing residue thiol protecting group is selectively deprotected while the peptide is attached to the resin. In this case, the peptide conjugate is formed through thioacylation of the free thiol group with a carboxylic acid of the lipid moiety.

Coupling of the peptide and the lipid moiety to form the peptide conjugate having a thioester linkage may be achieved by acylation or thioacylation methods known in the art. For example, the free thiol group of the thio-containing amino acid residue may be reacted with an activated carboxylic acid, such as an acid chloride or anhydride, on the lipid moiety. Alternatively, the thiol group of the thiol-containing amino acid residue may be reacted with the carboxylic acid of the lipid moiety in the presence of a coupling agent such as those described above in relation to amide bond formation during peptide synthesis, especially BOP.

During synthesis of the peptides that contain a free amino group, if required the amino group may be protected with a protecting group that may be selectively removed in the presence of other side chain protecting groups. For example, if the free amino group is present in a lysine introduced into the peptide, the free amino group may be protected during synthesis and selectively deprotected in the presence of other side chain or N-terminal protecting groups to allow selective formation of an amide bond with the lipid. Alternatively, if the free amino group is the N-terminal amino group, the N-terminal protecting group present during synthesis may be selectively deprotected in the presence of other protecting groups for selective formation of the amide bond with the lipid. Suitable amino protecting groups include Fmoc and BOC. The amino protecting group may need to be selected having consideration of other amino protecting groups used in the peptide.

Coupling of the amine-containing peptide and the lipid moiety to form the peptide conjugate having an amide linkage may be achieved by N-acylation methods known in the art. For example, the free amino group of the peptide may be reacted with an activated carboxylic acid of the lipid, such as an acid chloride or an anhydride. Alternatively the free amino group of the peptide may be reacted with the carboxylic acid of the lipid moiety in the presence of a coupling agent such as those described above in relation to amide bond formation during peptide synthesis.

In the case of solid phase synthesis, the peptide and lipid may be reacted together to form the amide linkage while the peptide is still attached to the resin.

Peptides may be cleaved from the resin used in the solid phase synthesis using standard techniques known in the art. For example, for cleavage of peptides prepared with Fmoc chemistry TFA or TFA compositions such as 95% TFA, 2.5% triisopropylsilane (TIS) AND 2.5% H₂O may be used. This also results in concomitant deprotection of amino acid side chains. For peptides where BOC chemistry is used HF may be used for resin cleavage and removal of side chain protecting groups.

The peptides or peptide conjugates may be isolated by precipitation and lyophylisation. Purification, if required, may be performed using reverse phase high performance liquid chromatography (RP-HPLC) and analysis may be performed using mass spectrometry, such as electrospray ionisation mass spectrometry.

### 6. Compositions

The present invention also contemplates compositions, including immunosuppressive compositions such as vaccines, comprising the peptide conjugate of the present invention as the active ingredient in suppressing or otherwise inhibiting an unwanted or undesirable immune response.

Also contemplated are compositions, including immunosuppressive compositions such as vaccines, consisting essentially of the peptide conjugate of the present invention as the active ingredient in suppressing or otherwise inhibiting an unwanted or undesirable immune response.

Also contemplated are compositions, including immunosuppressive compositions such as vaccines, consisting of the peptide conjugate of the present invention as the active ingredient in suppressing or otherwise inhibiting an unwanted or undesirable immune response.

In some embodiments these compositions may further comprise pharmaceutically acceptable excipients or diluents that are compatible with the active ingredient.

Suitable excipients or diluents are those which are non-toxic to the individual receiving the composition, including, for example, water, isotonic saline with or without a physiologically compatible buffer like phosphate or HEPES, dextrose, glycerol, ethanol, or the like and combinations thereof. Carrying reagents, such as albumin and blood plasma fractions and nonactive thickening agents, may also be used. Non-active biological components, to the extent that they are present in the composition, are especially derived from a syngeneic animal or human as that that will receive the composition, and are even more especially obtained previously from the subject to receive the composition.

In addition, if desired, the compositions may contain minor amounts of auxiliary substances such as wetting or emulsifying agents, pH buffering agents, and/or adjuvants that enhance the effectiveness of the immunosuppressive composition.

Examples of adjuvants which may be effective include but are not limited to: surface active substances such as hexadecylamine, octadecylamine, octadecyl amino acid esters, lysolecithin, dimethyldioctadecylammonium bromide, N,N-dicoctadecyl-N′, N′ bis(2-hydroxyethyl-propanediamine), methoxyhexadecylglycerol, and pluronic polyols; polyamines such as pyran, dextransulfate, poly IC carbopol; mineral gels such as aluminum phosphate, aluminum hydroxide or alum; peptides such as muramyl dipeptide and derivatives such as N-acetyl-muramyl-L-threonyl-D-isoglutamine (thur-MDP), N-acetyl-nor-muramyl-L-alanyl-D-isoglutamine (CGP 11637, referred to as nor-MDP), and N-acetylmuramyl-L-alanyl-D-isoglutaminyl-L-alanine-2-(1′-2′-dipalmitoyl-sn-glycero-3-hydroxyphosphoryloxy)-ethylamine (CGP 1983A, referred to as MTP-PE); lymphokines; QuilA and immune stimulating complexes (ISCOMS).

In some embodiments the composition may comprise a peptide conjugate of the present invention in the form of a pharmaceutically acceptable salt, including acid addition salts (formed with free amino groups of the peptide) which are formed with inorganic acids such as, for example, hydrochloric or phosphoric acids, or such organic acids such as acetic, oxalic, tartaric, maleic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The preparation of the compositions of the present invention uses routine methods known to persons skilled in the art. Techniques for formulation and administration of the compositions of the present invention may be found, for example, in “Remington's Pharmaceutical Sciences,” Mack Publishing Co., Easton, Pa., latest edition.

In some embodiments the compositions of the present invention are prepared as injectables, either as liquid solutions or suspensions, including vaccines. Solid forms suitable for solution in, or suspension in, liquid prior to injection may also be prepared.

Suitable administration routes may, for example, include oral, rectal, transmucosal, or intestinal administration; parenteral delivery, including intramuscular, intradermal, transdermal, subcutaneous, intramedullary, intrathecal, direct intraventricular, intravenous, intraperitoneal, intranasal, or intraocular injections.

If desired, devices or pharmaceutical compositions or compositions containing the peptide conjugate and suitable for sustained or intermittent release could be, in effect, implanted in the body or topically applied thereto for the relatively slow release of the peptide conjugate or composition of the invention into the body.

### 7. Methods for Modulating Immune Responses

The present invention also extends to methods for suppressing or otherwise inhibiting an unwanted or undesired immune response, including immune responses to a target antigen, in a subject by administering the peptide conjugates or compositions of the present invention. In some embodiments, the immune response is a T-cell mediated response. In some other embodiments, the immune response is an antibody-mediated response.

Also encapsulated by the present invention is a method for preventing, inhibiting, treating or decreasing an autoimmune, an allergic immune or an allograft immune response in a subject comprising administering an effective amount of a peptide conjugate or a composition of the present invention.

The method may be performed before or once the subject displays symptoms of the immune response, including administering to the subject before or after the onset of symptoms. In some embodiments, treatment commences 1 day, 2 days, 3 days, 4 days, 5 days, 6 days, 10 days, 1 month, 2 months, 6 months, 8 months, 12 months or 18 months after the onset of symptoms of the immune response. As used herein, the term “onset of symptoms” includes the first time the subject has displayed any symptom of the immune response, or may be at a time when the subject displays symptoms of the immune response following a remission period where the subject did not display symptoms of the immune response following a period of displaying symptoms of the immune response. In some embodiments, treatment commences when the subject is in remission, i.e. when the subject has previously displayed symptoms of the immune response but is not currently displaying symptoms of the immune response.

Autoimmune responses that may be prevented, inhibited, treated or decreased by the present invention include tissue specific or systemic autoimmune diseases, including, but not limited to, Psoriasis, Acute disseminated encephalomyelitis (ADEM), Hashimoto's thyroiditis, Addison's disease, Idiopathic thrombocytopenic purpura, Ankylosing spondylitis, Lupus erythematosus, Antiphospholipid antibody syndrome (APS), Multiple sclerosis (MS), Autoimmune haemolytic anemia, Myasthenia gravis, Autoimmune hepatitis, Pemphigus, Bullous pemphigoid, Pernicious anaemia, Churg-Strauss Syndrome (or allergic granulomatosis), Polymyositis, Coeliac disease, Primary biliary cirrhosis, Dermatomyositis, Rheumatoid arthritis, Diabetes mellitus type 1 (IDDM), Sjögren's syndrome, Goodpasture's syndrome, Temporal arteritis (or “giant cell arteritis”), Graves' disease, Vasculitis, Guillain-Barrë syndrome (GBS), Wegener's granulomatosis, scleroderma and chronic inflammatory demyelinating polyradiculoneuropathy (CIDP).

Allergic immune responses that may be prevented, inhibited, treated or decreased by the present invention include, but are not limited to both type I and type IV hypersensitivity immune responses such as hayfever, skin inflammation (urticaria), food allergies, asthma and systemic anaphylaxis, dermatitis, tuberculin reaction, and chronic transplant rejection.

Allograft immune responses that may be prevented, inhibited, treated or decreased by the present invention include any unwanted or undesired immune response in a subject as the result of allograft transplantation to the subject. Suitable allograft transplants include organs and tissue including heart, lung, kidney, liver, pancreas, intestine, skin and stem cells, including hematopoietic stem cells and mesenchymal stem cells.

### 8. In Vivo Administration

In some embodiments, the methods of the present invention may be achieved by in vivo administration of the peptide conjugates or compositions of the present invention as described above.

The dosage of the peptide conjugate or composition of the present invention to be administered may depend on the subject to receive the peptide conjugate or composition inclusive of the age, sex, weight and general health condition thereof. The dosage will also take into consideration the pharmacokinetic properties of the peptide including the binding affinity of MHC class II molecules or MHC class I molecules to the peptide and the binding affinity of the T cell receptors to the peptide, the ability of the peptide conjugate to enter antigen presenting cells and pharmacokinetics compared to any antigen which is competing for the same MHC molecules and T cell receptor sites. In this regard, precise amounts of the peptide conjugate for administration can also depend on the judgment of the practitioner.

In determining the effective amount of the peptide conjugate or composition to be administered in the prevention, inhibition, treatment or decrease of an autoimmune, an allergic immune or an allograft immune response in a subject, the physician or veterinarian may evaluate the subject's predisposition to the autoimmune, allergic immune or allograft immune response, or the progression of the immune response over time. In any event, those of skill in the art may readily determine suitable dosages of the agents of the invention without undue experimentation.

The dosage of the peptide conjugate or composition administered to a patient should be sufficient to effect a beneficial response in the patient over time such as a reduction in the symptoms, reduced or no relapses in a patient, or the exhibition of symptoms or other parameters that indicate a beneficial response. For example, a reduction in MRI lesions in a subject with multiple sclerosis is generally accepted in clinical trials as indicating a beneficial response to the treatment. Examples of usual patient dosages for systemic administration of the peptide conjugate stated in terms of patient body weight range from about 0.005-1.0 mg/kg, typically from about 0.01-0.5 mg/kg, more typically from about 0.05-0.1.

The dosages may be administered at suitable intervals such as to maintain an immunosuppressive effect, or boost the immunosuppressive response. Such intervals can be ascertained using routine procedures known to persons of skill in the art and can vary depending on the type of peptide conjugate employed and its formulation. For example, the interval may be daily, every other day, weekly, fortnightly, monthly, bimonthly, quarterly, half-yearly or yearly.

### 9. Ex Vivo Administration

In other embodiments, peptide conjugates or compositions of the present invention may be contacted ex vivo with antigen presenting cells or their precursors and the resulting mixture administered to the subject.

In some ex vivo administration embodiments, the antigen presenting cells or precursors are derived or obtained from the subject receive the peptide conjugate or composition (i.e. an autologous antigen presenting cells). In other ex vivo administration embodiments, the antigen presenting cells or precursors are derived or obtained from a donor that is MHC matched or mismatched with the subject (i.e., an allogeneic antigen presenting cells). Suitably, the donor is histocompatible with the subject. In these embodiments, the antigen presenting cell is contacted with at least one peptide conjugate of the present invention which is suitably in soluble form in an amount and for a time sufficient for the peptide conjugate to be processed and the peptide (or processed form thereof) presented by the antigen presenting cells on their surface. Suitably the peptide conjugate is part of an immunomodulating composition including those described above.

9.1 Sources of Antigen Presenting Cells and their Precursors

Antigen presenting cells or their precursors can be isolated by methods known to those of skill in the art. The source of such cells will differ depending upon the antigen presenting cell required for modulating a specified immune response. In this context, the antigen presenting cell can be selected from dendritic cells, macrophages, monocytes and other cells of myeloid lineage.

Typically, precursors of antigen presenting cells can be isolated from any tissue, but are most easily isolated from blood, cord blood or bone marrow (Sorg et al., 2001; Zheng et al., 2000). It is also possible to obtain suitable precursors from diseased tissues such as rheumatoid synovial tissue or fluid following biopsy or joint tap (Thomas et al., 1994a; Thomas et al., 1994b). Other examples include, but are not limited to liver, spleen, heart, kidney, gut and tonsil (Lu et al., 1994; McIlroy et al., 2001; Vremec et al., 2000; Hart and Fabre, 1981; Hart and McKenzie, 1988; Pavli et al., 1990).

Leukocytes isolated directly from tissue provide a major source of antigen presenting cell precursors. Typically, these precursors can only differentiate into antigen presenting cells by culturing in the presence or absence of various growth factors. According to the practice of the present invention, the antigen presenting cells may be so differentiated from crude mixtures or from partially or substantially purified preparations of precursors. Leukocytes (peripheral blood mononuclear cells or PBMCs) can be conveniently purified from blood or bone marrow by density gradient centrifugation using, for example, Ficoll Hypaque which eliminates neutrophils and red cells, or by ammonium chloride lysis of red cells (leukocytes or white blood cells). Many precursors of antigen presenting cells are present in peripheral blood as non-proliferating monocytes, which can be differentiated into specific antigen presenting cells, including macrophages and dendritic cells, by culturing in the presence of specific cytokines.

Tissue-derived precursors such as precursors of tissue dendritic cells or of Langerhans cells are typically obtained by mincing tissue (e.g., basal layer of epidermis) and digesting it with collagenase or dispase followed by density gradient separation, or selection of precursors based on their expression of cell surface markers. For example, Langerhans cell precursors express CD1 molecules as well as HLA-DR and can be purified on this basis.

In some embodiments, the antigen presenting cell precursor is a precursor of macrophages. Generally these precursors can be obtained from monocytes of any source and can be differentiated into macrophages by prolonged incubation in the presence of medium and macrophage colony stimulating factor (M-CSF) (Erickson-Miller et al., 1990; Metcalf and Burgess, 1982).

In other embodiments, the antigen presenting cell precursor is a precursor of Langerhans cells. Usually, Langerhans cells can be generated from human monocytes or CD34⁺ bone marrow precursors in the presence of granulocyte/macrophage colony-stimulating factor (GM-CSF), IL-4/TNFα and TGFβ (Geissmann et al., 1998; Strobl et al., 1997a; Strobl et al., 1997b; Strobl et al., 1996).

In still other embodiments, the antigen presenting cell precursor is a precursor of dendritic cells. Several potential dendritic cell precursors can be obtained from peripheral blood, cord blood or bone marrow. These include monocytes, CD34⁺ stem cells, granulocytes, CD33⁺CD11c⁺ DC precursors, and committed myeloid progenitors—described below.

9.2 Monocytes

Monocytes can be purified by adherence to plastic for 1-2 hours in the presence of tissue culture medium (e.g., RPMI) and serum (e.g., human or foetal calf serum), or in serum-free medium (Anton et al., 1998; Araki et al., 2001; Mackensen et al., 2000; Nestle et al., 1998; Romani et al., 1996; Thurner et al., 1999). Monocytes can also be elutriated from peripheral blood (Garderet et al., 2001). Monocytes can also be purified by immunoaffinity techniques, including immunomagnetic selection, flow cytometric sorting or panning (Araki et al., 2001; Battye and Shortman, 1991), with anti-CD14 antibodies to obtain CD14hi cells. The numbers (and therefore yield) of circulating monocytes can be enhanced by the in vivo use of various cytokines including GM-CSF (Groopman et al., 1987; Hill et al., 1995). Monocytes can be differentiated into dendritic cells by prolonged incubation in the presence of GM-CSF and IL-4 (Romani et al., 1994; Romani et al., 1996). A combination of GM-CSF and IL-4 at a concentration of each at between about 200 to about 2000 U/mL, more preferably between about 500 to about 1000 U/mL and even more preferably between about 800 U/mL (GM-CSF) and 1000 U/mL (IL-4) produces significant quantities of immature dendritic cells, i.e., antigen-capturing phagocytic dendritic cells. Other cytokines which promote differentiation of monocytes into antigen-capturing phagocytic dendritic cells include, for example, IL-13.

9.3 CD34⁺ Stem Cells

Dendritic cells can also be generated from CD34⁺ bone marrow derived precursors in the presence of GM-CSF, TNFα±stem cell factor (SCF, c-kitL), or GM-CSF, IL-4±flt3L (Bai et al., 2002; Chen et al., 2001; Loudovaris et al., 2001). CD34⁺ cells can be derived from a bone marrow aspirate or from blood and can be enriched as for monocytes using, for example, immunomagnetic selection or immunocolumns (Davis et al., 1994). The proportion of CD34⁺ cells in blood can be enhanced by the in vivo use of various cytokines including (most commonly) G-CSF, but also flt3L and progenipoietin (Fleming et al., 2001; Pulendran et al., 2000; Robinson et al., 2000).

9.4 Other Myeloid Progenitors

DC can be generated from committed early myeloid progenitors in a similar fashion to CD34⁺ stem cells, in the presence of GM-CSF and IL-4/TNF. Such myeloid precursors infiltrate many tissues in inflammation, including rheumatoid arthritis synovial fluid (Santiago-Schwarz et al., 2001). Expansion of total body myeloid cells including circulating dendritic cell precursors and monocytes, can be achieved with certain cytokines, including flt-3 ligand, granulocyte colony-stimulating factor (G-CSF) or progenipoietin (pro-GP) (Fleming et al., 2001; Pulendran et al., 2000; Robinson et al., 2000). Administration of such cytokines for several days to a human or other mammal would enable much larger numbers of precursors to be derived from peripheral blood or bone marrow for in vitro manipulation. Dendritic cells can also be generated from peripheral blood neutrophil precursors in the presence of GM-CSF, IL-4 and TNFα (Kelly et al., 2001; Oehler et al., 1998). It should be noted that dendritic cells can also be generated, using similar methods, from acute myeloid leukaemia cells (Oehler et al., 2000).

9.5 Tissue DC Precursors and Other Sources of APC Precursors

Other methods for DC generation exist from, for example, thymic precursors in the presence of IL-3+/−GM-CSF, and liver DC precursors in the presence of GM-CSF and a collagen matrix. Transformed or immortalised dendritic cell lines may be produced using oncogenes such as v-myc or by myb.

9.6 Circulating DC Precursors

These have been described in human and mouse peripheral blood. One can also take advantage of particular cell surface markers for identifying suitable dendritic cell precursors. Specifically, various populations of dendritic cell precursors can be identified in blood by the expression of CD11c and the absence or low expression of CD14, CD19, CD56 and CD3 (O'Doherty et al., 1994; O'Doherty et al., 1993). These cells can also be identified by the cell surface markers CD13 and CD33 (Thomas et al., 1993). A second subset, which lacks CD14, CD19, CD56 and CD3, known as plasmacytoid dendritic cell precursors, does not express CD11c, but does express CD123 (IL-3R chain) and HLA-DR (Farkas et al., 2001; Grouard et al., 1997; Rissoan et al., 1999). Most circulating CD11c⁺ dendritic cell precursors are HLA-DR⁺, however some precursors may be HLA-DR-. The lack of MHC class II expression has been clearly demonstrated for peripheral blood dendritic cell precursors (del Hoyo et al., 2002).

Optionally, CD33⁺CD14⁻/lo or CD11c⁺HLA-DR⁺, lineage marker-negative dendritic cell precursors described above can be differentiated into more mature antigen presenting cells by incubation for 18-36 h in culture medium or in monocyte conditioned medium (Thomas et al., 1993; Thomas and Lipsky, 1994; O'Doherty et al., 1993). Alternatively, following incubation of peripheral blood non-T cells or unpurified PBMC, the mature peripheral blood dendritic cells are characterised by low density and so can be purified on density gradients, including metrizamide and Nycodenz (Freudenthal and Steinman, 1990; Vremec and Shortman, 1997), or by specific monoclonal antibodies, such as but not limited to the CMRF-44 mAb (Fearnley et al., 1999; Vuckovic et al., 1998). Plasmacytoid dendritic cells can be purified directly from peripheral blood on the basis of cell surface markers, and then incubated in the presence of IL-3 (Grouard et al., 1997; Rissoan et al., 1999). Alternatively, plasmacytoid DC can be derived from density gradients or CMRF-44 selection of incubated peripheral blood cells as above.

In general, for dendritic cells generated from any precursor, when incubated in the presence of activation factors such as monocyte-derived cytokines, lipopolysaccharide and DNA containing CpG repeats, cytokines such as TNF-α, IL-6, IFN-α, IL-1β, necrotic cells, re-adherence, whole bacteria, membrane components, RNA or polyIC, immature dendritic cells will become activated (Clark, 2002; Hacker et al., 2002; Kaisho and Akira, 2002; Koski et al., 2001). This process of dendritic cell activation is inhibited in the presence of NF-κB inhibitors (O'Sullivan and Thomas, 2002).

9.7 Ex Vivo Delivery of Peptide Conjugates

The amount of peptide conjugate to be placed in contact with antigen presenting cells can be determined empirically by routine methods known to persons of skill in the art. Typically antigen presenting cells are incubated with the peptide conjugate of the present invention for about Ito 6 hours at 37° C., although it is also possible to expose antigen presenting cells to peptide conjugate for the duration of incubation with growth factors. Generally, 0.001-1000 μg/mL of peptide conjugate is suitable for producing antigen presenting cells that are presenting the peptides on their surface through MHC I or MHC II molecules. Usually 0.01-100 μg/mL of peptide conjugate is suitable for producing antigen presenting cells that are presenting the peptides on their surface through MHC I or MHC II molecules. Typically, 0.1-10 μg/mL of peptide conjugate is suitable for producing antigen presenting cells that are presenting the peptides on their surface through MHC I or MHC II molecules.

The antigen presenting cells should be exposed to the peptide conjugate for a period of time sufficient for those cells to internalise the peptide. Advantageously the time should be sufficient so that the antigen presenting cell also presents the peptide of the peptide conjugate or a processed form thereof on its surface. The time and dose of peptide conjugate necessary for the cells to internalise and present the processed peptide may be determined using pulse-chase protocols in which exposure to peptide conjugate is followed by a washout period and exposure to a read-out system e.g., T cells reactive to the original antigen upon which the peptide of the peptide conjugate is based. Once the optimal time and dose necessary for cells to express processed peptide on their surface is determined, a protocol may be used to prepare cells and peptide conjugate for suppressing or otherwise inhibiting an unwanted or undesired immune responses. Those of skill in the art will recognise in this regard that the length of time necessary for an antigen presenting cell to process and present the peptide will vary depending on the peptide or form of peptide in the peptide compound employed, its dose, and the antigen presenting cell employed, as well as the conditions under which peptide loading is undertaken. These parameters can be determined by the skilled artisan using routine procedures.

The antigen presenting cells can be introduced into a patient by any means (e.g., injection), which produces the desired immunosuppressive response to a peptide. The cells may be derived from the patient (i.e., autologous cells) or from an individual or individuals who are MHC-matched or -mismatched (i.e., allogeneic) with the patient. In specific embodiments, autologous cells are injected back into the patient from whom the source cells were obtained. The injection site may be subcutaneous, intraperitoneal, intramuscular, intradermal, or intravenous. The cells may be administered to a patient already suffering from the unwanted immune response or who is predisposed to the unwanted immune response in sufficient number to prevent or at least partially arrest the development, or to reduce or eliminate the onset of, that response. The number of cells injected into the patient in need of the treatment or prophylaxis may vary depending on inter alia, the peptide conjugate and size of the individual. This number may range for example between about 10³ and 10¹¹, and more preferably between about 10⁵ and 10⁷ cells (e.g., dendritic cells). Single or multiple administrations of the cells can be carried out with cell numbers and pattern being selected by the treating physician. The cells should be administered in a pharmaceutically acceptable excipient, which is non-toxic to the cells and the individual. Such excipient may be the growth medium in which the cells were grown, or any suitable buffering medium such as phosphate buffered saline. The cells may be administered alone or as an adjunct therapy in conjunction with other therapeutics known in the art for the treatment or prevention of unwanted immune responses for example but not limited to glucocorticoids, methotrexate, D-penicillamine, hydroxychloroquine, gold salts, sulfasalazine, TNFα or interleukin-1 inhibitors, and/or other forms of specific immunotherapy.

In order that the invention may be readily understood and put into practical effect, particular preferred embodiments will now be described by way of the following non-limiting examples.

### EXAMPLES

### Example 1

### Peptide and Peptide Conjugate Synthesis

### Materials and Methods

Fmoc-1-amino acids were purchased from Nova biochem (Meudon, France) or Neosystem (strasbourg, France). Preloaded Wang-resin was obtained from Novabiochem and BOP from Neosystem. Palmitoyl chloride (Pam-Cl) and palmitic acid (Pam-OH) were purchased from Fluka (St-Quentic Fallavier, France). Dimethylformamide (DMF) was purchased from Merck (Briare Le Canal, France).

### Peptide Synthesis

The peptide (0.2 mmol) was synthesised manually on a Wang resin using the Fmoc/tBu strategy and BOP as coupling reagent. Typically, successive single couplings were performed with three equivalents of Fmoc amino acid and were monitored with the Kaiser colour test. Fmoc amino acids side-chain protecting groups were Lys(Dde), Asp(OtBu), His(Trt), Trp(Boc), Lys(Boc), Cys(Mmt). Boc-His(Boc)-OH was coupled as N-terminal amino acid and obtained by conversion of its DCHA salt.

### Deprotection of S-Mmt Cysteinyl Residue

The peptide resin (300 mg) was treated with a solution of 2% TFA in dichloromethane (DCM) containing 5% TIS for 10 min in a glass reaction vessel equipped with a sintered glass filter using nitrogen for mixing. After filtration, the peptide-resin was washed with DCM. The deprotection and washing steps were repeated five times. The peptide-resin was finally washed with DCM.

### Deprotection of S-(StBu) Cysteinyl Residue

Peptide resin (50 mg) was introduced into a round bottom flask equipped with a condenser. A freshly prepared mixture of β-mercaptoethanol/DMF (1/1, v/v) (5 ml) was added. The mixture was heated overnight at 85° C. After filtration, the peptide resin was washed with DMF.

Another example of carrying out deprotection of S-(tBu) cysteinyl residue is to use tributylphosphine. In this alternative means, the peptide resin (50 mg) may be treated with a solution of tributylphosphine (100 equiv) and H2O (400 equiv) in DMF:DCM (1/1, 3 ml) for 18 hours in a glass reaction vessel equipped with a sintered glass filter using nitrogen for mixing. After filtration, the peptide-resin may be washed with DCM and DMF.

Palmitoylation of Free Cysteinyl Thiol Group with Palmitoyl Chloride

In a glass reaction vessel equipped with a sintered glass filter, the biotinylated free thiol peptide resin (50 mg) was suspended in DMF (2 mL) containing palmitoyl chloride (20 eq). Dimethyl amino pyridine (DMAP) (0.1 eq) dissolved in distilled pyridine (2.5 mL) was then added. After 1 hour of reaction at room temperature, the peptide resin was filtered off, washed with DMF, DCM, ether, and then dried in a vacuum.

Palmitoylation of Free Cysteinyl Thiol Group with Palmitic Acid

In a glass reaction vessel equipped with a sintered glass filter, the biotinylated free thiol peptide resin (300 mg) was suspended in DMF (3 ml) containing palmitic acid (20 eq) and BOP (20 eq). Diisopropylethylamine (DIEA) (60 eq) was then added. After 1 hour of reaction at room temperature, the peptide resin was filtered off, washed with DMF, DCM, ether and then dried in a vacuum.

### Palmitoylation of the N-Terminal Amino Group

Deprotection of the N-terminal Fmoc protecting group was achieved with 20% piperidine in DMF and treatment of the deprotected N-terminal amino group with palmitic acid in the presence of BOP. DIEA was then added. After 1 hour reaction at room temperature the peptide-resin was filtered off and washed with DMF, DCM and ether and dried under vacuum.

Cleavage from Resin

Peptides were cleaved from the resin with the low odor mixture (95% trifluoroacetic acid (TFA), 2.5% triisopropylsilane (TIS), 2.5% H2O). After evaporation of TFA, peptides were precipitated in ethyl ether and lyophilised after solubilisation in 10% acetic acid.

By the above methods, the following peptide conjugates, palmitoylated proteolipid proteins and peptides were prepared:

PLP139-151 (W144)
SEQ ID NO: 1
H₂N-HCLGKWLGHPDKF-CO₂H
Npalm-PLP139-151
SEQ ID NO: 2
(palm)HN-HCLGKWLGHPDKF
Spalm-PLP 139-151
SEQ ID NO: 3
H₂N-HC(palm)LGKWLGHPDKF-CO₂H
APL (Q144)
SEQ ID NO: 4
H₂N-HCLGKQLGHPDKF-CO₂H
lipoAPL (Spalm-Q144)
SEQ ID NO: 5
H₂N-HC(palm)LGKQLGHPDKF-CO₂H
PLP178-191 (F188)
SEQ ID NO: 6
H₂N-NTWTTCQSIAFPSK-CO₂H
Spalm-PLP178-191
SEQ ID NO: 7
H₂N-NTWTTC(palm)QSIAFPSK-CO₂H
APL (A188)
SEQ ID NO: 8
H₂N-NTWTTCQSIAAPSK-CO₂H
lipoAPL (Spalm A188)
SEQ ID NO: 9
H₂N-NTWTTC(palm)QSIAAPSK-CO₂H
PLP104-117
SEQ ID NO: 10
H₂N-KTTICGKGLSATVT-CO₂H
Spalm-PLP 104-117
SEQ ID NO: 11
H₂N-KTTIC(palm)GKGLSATVT-CO₂H
Npalm-PLP 104-117
SEQ ID NO: 12
(palm)HN-KTTICGKGLSATVT

### Example 2

### Multiple Sclerosis (MS) Animal Model

MS is a chronic inflammatory demyelinating and neurodegenerative disease of the central nervous system (CNS). It is a CD4⁺ mediated disease and has a strong association with MHC class II. The pathogenesis of MS is not fully elucidated, but studies conducted with the animal model of MS, experimental autoimmune encephalomyelitis (EAE), has provided insight into how the immune system can provoke an immunopathological response characteristic of that seen in MS.

EAE can be induced in animal by injection of myelin proteins, such as MBP, PLP (proteolipid protein, the major protein of myelin CNS) and MOG, or their encephalitogenic peptides emulsified in CFA (complete Freund's adjuvant). EAE is also a useful model for aiding the development of new prophylaxis or treatment methods for MS. All therapies approved for MS ameliorate EAE, and two approved medications: glatiramer acetate and Natalizumab, were developed directly from studies in EAE.

The acylation sites Cys108, Cys140, and Cys183 are within the encephalitogenic PLP epitopes PLP104-117, PLP139-151, and PLP178-191 respectively (Greer et al. 1996; Greer et al., 2001). Reactivity to these epitopes has been found in some patients with MS.

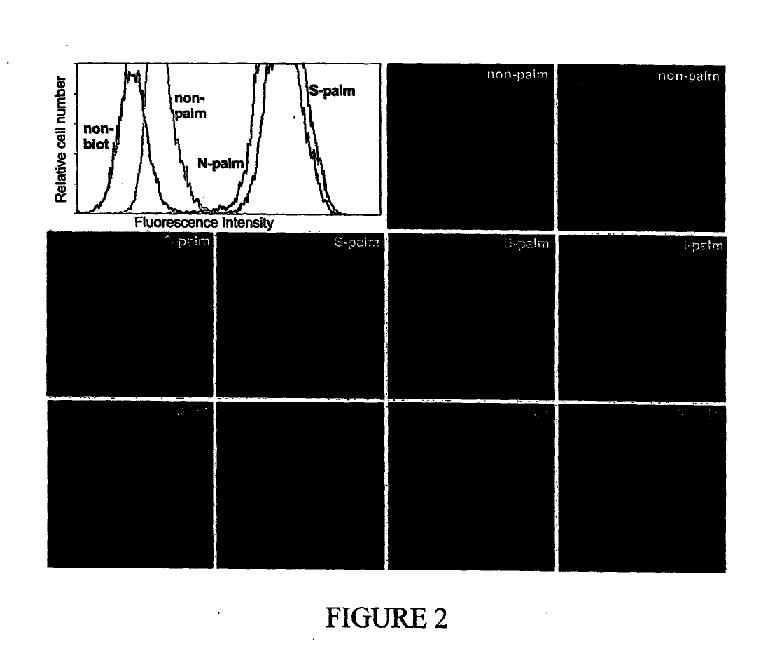

In examples 2 and 4-8, PLP encephalitogenic epitopes were injected into SJL/J mice to induce EAE, so the mice could be used as an animal model for MS. Such a model has been used in numerous studies, including where preventative or treatment methods for MS are being investigated. The study described in Hofstetter et al., 2007, for example, describes the induction of EAE in SJL mice at pages 1373-1374. FIG. 2 in that paper shows the clinical course of PLPp-induced EAE in the SJL colony used by the authors, using a 5 point severity scale similar to that used in the examples below. The Figure shows a zero mean score until day 10 (after PLPp immunisation) when the mean score demonstrates the start of signs the disease in the mice, peaking at approximately day 20 (after PLPp immunisation), following by a decrease in mean score until approximately day 38 and slight increase to what appears to be a constant mean score of 1 for the next 20 days.

The authors of Hofstetter et al. note that prior to onset of the disease, the mice are described as being in “pre-EAE onset stage” including at day 8 where PLPp induced IL-17-producing cells were detected in the drLN and in the spleen but not in the CNS. The authors also note that following immunisation with PLPp, >90% of the mice developed EAE with the onset of disease occurring between days 11 and 12. At day 12, during acute EAE, the authors state that PLPp-specific IL-17 producing cells became detectable in the CNS, occurring there in higher frequencies than in the drLN and spleen.

### Method

In this example, the following peptides produced in Example 1 were used:

Non-palmitoylated peptides

| | |
|---|---|
| PLP104-117 | K-T-T-I-C-G-K-G-L-S-A-T-V-T |
| | (SEQ ID NO: 10) |
| PLP139-151 | H-C-L-G-K-W-L-G-H-P-D-K-F |
| | (SEQ ID NO: 1) |
| PLP178-191 | N-T-W-T-T-C-Q-S-I-A-F-P-S-K |
| | (SEQ ID NO: 6) |

S-palmitoylated peptides

| | |
|---|---|
| Spalm-PLP104- | K-T-T-I-C(palm)-G-K-G-L-S-A-T-V-T |
| 117 | (SEQ ID NO: 10) |
| Spalm-PLP139- | H-C(palm)-L-G-K-W-L-G-H-P-D-K-F |
| 151 | (SEQ ID NO: 3) |
| Spalm-PLP178- | N-T-W-T-T-C(palm)-Q-S-I-A-F-P-S-K |
| 191 | (SEQ ID NO: 7) |

N-palmitoylated peptides

| | |
|---|---|
| Npalm-PLP104- | (palm)HN-K-T-T-I-C-G-K-G-L-S-A- |
| 117 | T-V-T |
| | (SEQ ID NO: 10) |
| Npalm-PLP139- | (palm)HN-H-C-L-G-K-W-L-G-H-P- |
| 151 | D-K-F |
| | (SEQ ID NO: 3) |

Mice were immunised with the above non-palmitoylated, S-palmitoylated or N-palmitoylated peptides in complete Freund's adjuvant to induce EAE.

(1) Mice were followed for 6 weeks to determine the induction of EAE.

Lymph nodes from a different group of mice immunised in the same manner were removed 10 days after injection of the peptides and single cell suspensions of lymph node cells (LNC) were prepared.

(2) Some of the LNC were tested for their ability to proliferate in response to peptide stimulation in tritiated thymidine uptake assays. LNC (3×10⁵/well) were incubated at 37° C. for 3 days in 96 well plates with 0-50 μg/ml antigen in a total volume of 200 μl (diluted in RPMI 1640 medium containing 10% FCS). Tritiated thymidine was added during the last 18 h of culture. Cells were harvested onto glass fibre filters, which were then put through a beta counter. Increased uptake of tritiated thymidine into cells indicated proliferation of the cells.

(3) Other LNC were incubated with antibodies against CD4 or CD8, and the ratio of CD4 to CD8 was determined using flow cytometric techniques. LNC were centrifuged through a Ficoll gradient and washed with PBS containing 1% FCS and 0.01% sodium azide (wash buffer). Aliquots of 1 million cells were incubated with antibodies specific for CD4 (clone RM4-5; rat IgG2a) or CD8a (clone 53-6.7; rat IgG2a) together with an antibody specific for the TCR β-chain (H57-597; hamster IgG) for 30 min at 4° C. in the dark, followed by FITC-conjugate anti-rat κ-chain or PE-conjugated anti-hamster IgG for 30 min at 4° C. in the dark. Isotype-matched primary antibodies were used as controls. All antibodies were purchased from PharMingen (San Diego, Calif.) and were used at 1 μg/ml dilution in wash buffer. After washing, cells were resuspended in wash buffer and analysed using a FACSCalibur flow cytometer (Becton Dickinson, Franklin Lakes, N.J.).

### Results and Conclusions

Regarding (1), it was found that immunisation of mice with S-palmitoylated peptides enhanced the development and chronicity of EAE compared to the non-palmitoylated and N-palmitoylated peptides as shown in Table 3.

TABLE 3
Effect of different peptides on development and chronicity of EAE

| Peptide | Incidence | Day of Onset | Score | Duration |
|---|---|---|---|---|
| PLP₁₀₄₋₁₁₇ | 0/4 | | 0 | |
| S-palm-PLP₁₀₄₋₁₁₇^{a} | 4/4 | 40.5 ± 29.9 | 3.0 ± 0 | 7.7 ± 0.5 |
| N-palm-PLP₁₀₄₋₁₁₇ | 0/4 | | 0 | |
| PLP₁₃₉₋₁₅₁ | 4/4 | 11.0 ± 1.4 | 3.0 ± 0.7 | 5.8 ± 1.8 |
| S-palm-PLP₁₃₉₋₁₅₁ | 4/4 | 11.0 ± 0 | 4.3 ± 0.5 | 12.8 ± 2.8^{b} |
| N-palm-PLP₁₃₉₋₁₅₁ | 0/4 | | 0 | |
| PLP₁₇₈₋₁₉₁ | 5/5 | 13.0 ± 1.5 | 2.7 ± 0.9 | 5.8 ± 0.4 |
| S-palm-PLP₁₇₈₋₁₉₁ | 5/5 | 10.6 ± 0.5 | 3.5 ± 0.3 | 9.6 ± 1.0 |

^{a}The incidence of disease and clinical score for mice immunised with S-palm-PLP104-117 are significantly different from those for mice immunised with PLP104-117 (p ≦ 0.001).
^{b}The duration of the first episode of disease in mice immunised with S-palm-PLP139-151 is significantly different than that of mice immunised with PLP139-151 (p ≦ 0.007).

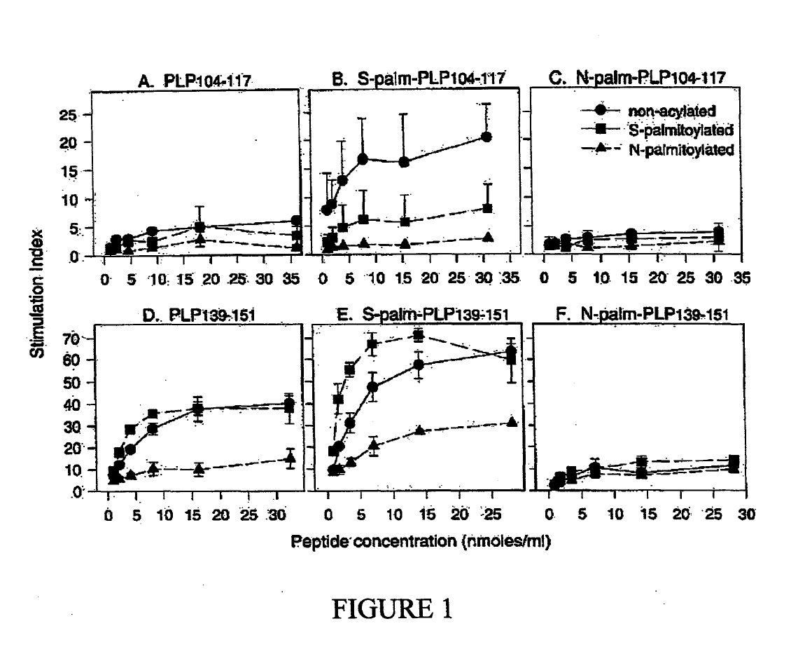

Regarding (2), it was found that the LNC taken from mice immunised with S-palmitoylated peptides induced a greater CD4⁺ T cell response when incubated with the PLP peptide as shown in FIG. 1. FIG. 1 is a graphical representation showing the proliferative responses of the LNC to the nonacylated and acylated PLP peptides. The peptide with which the mice had been immunised is shown above each graph. Each graph shows the proliferative responses of the LNC to the nonacylated (circle), S-palm (square), or N-palm (triangle) form of the same peptide. Each point on the graph represents the SI (mean±SD of three to five repetitions of each experiment) at a particular peptide concentration.

Regarding (3), it was found that the mice immunised with the N-palmitoylated peptides induced a T cell response with a decreased CD4:CD8 ratio as shown in Table 4, and were not encephalitogenic.

TABLE 4
CD4/CD8 ratios of activated LNC from mice (4 per group)
immunised with nonacylated or palmitoylated peptides

| LNC from Mice Immunised with | CD4/CD8 Ratio (mean ± SEM) |
|---|---|
| PLP104-117 | 2.31 ± 0.07 |
| S-palm-PLP104-117 | 2.71 ± 0.16^{a} |
| N-palm-PLP104-117 | 2.07 ± 0.06^{b} |
| PLP139-151 | 2.18 ± 0.19 |
| S-palm-PLP139-151 | 2.46 ± 0.08^{c} |
| N-palm-PLP139-151 | 1.96 ± 0.06 |

^{a}Ratio significantly different than that for LNC from mice immunised with PLP104-117 (p < 0.03) and N- palm-PLP104-117 (p < 0.003).
^{b}Ratio significantly different than that for LNC from mice immunised with PLP104-117 (p < 0.019).
^{c}Ratio significantly different than that for LNC from mice immunised with Npalm-PLP139-151 (p < 0.003).

Thus this example shows that S-palmitoylation (thiopalmitoylation) of the peptides increased the immunogenicity and the encephalitogenicity of the peptides and induces a CD4⁺ (or class II) response.

### Example 3

### Enhancement of Uptake into Antigen-Presenting Cells by Acylation of Peptides

The mechanism underlying the enhancement of CD4 (class II responses) by S-palm peptides compared to non-acylated and N-palm peptides was investigated further.

### Method

For confocal microscopy, macrophages were isolated from normal 8-12-wk old female SJL/J mice peritoneal cell suspensions by adhesion to glass coverslips. The macrophages were then incubated with 100 μM peptide for between 1 min and 24 h at 37° C., the peptide being one of:

| | |
|---|---|
| PLP139-151 | (nonbiotinylated, nonpalm) |
| PLP139-151 | Cys140-SH Lys150-Biot (nonpalm) |
| PLP139-151 | Cys140-Palm Lys150-Biot (S-palm) |
| PLP139-151 | His139-Palm Lys150-Biot (N-palm) |

After incubation, cells were fixed with 4% formaldehyde in PBS for 15 min at room temperature and permeabilised with 0.05% digitonin in PBS for 5 min at room temperature or 0.2% Triton X-100 in PBS for 20 min at room temperature.

The cells were then incubated with murine antibodies against endosomes, lysosomes, endoplasmic reticulum, or MHC class I or II molecules for 1-3 hours at 37° C. After three washes in PBS, cells were incubated with streptavidin-Alexa 488 (1/400 dilution in PBS) or streptavidin-Cy3 (1/450 dilution in PBS) (both from Molecular Probes) and Texas Red-labeled rabbit anti-mouse IgG or IgM for 30 min in the dark at room temperature. After washing with PBS, coverslips were mounted in Aquapolymount medium. Immunofluorescence staining was monitored with a laser scanning microscope (LSM 510; Carl Zeiss Laboratories) equipped with a Plan-Apochromat 63× oil DIC immersion lens (numerical aperture 1.4). Alexa 488 emission was excited using the 488-nm ray of the argon laser, whereas Texas Red was excited using the 543-nm line of the helium/neon laser. Emission signals of Alexa 488 and Texas Red were filtered with a LP 505-530 and a LP 560 filter, respectively. Quantification of colocalisation was performed on cells from 10 to 12 fields (at least 100 cells) with the colocalisation module of the Zeiss LSM Image Browser software.

For flow cytometry, PC were washed and incubated with peptide for various times at 37 or 5° C. At the end of the incubation, they were fixed in freshly prepared 4% formaldehyde in PBS for 15 min at room temperature. After two washes with PBS, PC were permeabilised with 0.05% digitonin for 5 min at room temperature, washed twice more with PBS containing 1% FCS and 0.01% sodium azide, and double stained with PE-labeled F4/80 antibody to detect macrophages and FITC-streptavidin to detect the biotinylated peptide. After washing in PBS containing 1% FCS and 0.01% sodium azide, the cells were analysed by flow cytometry using a FacsCalibur system (BD Biosciences). Samples were gated on the F4/80⁺ population, and the mean fluorescence intensity of staining with FITC-streptavidin was determined

### Results and Conclusions

Uptake of the peptides into the macrophages was monitored by flow cytometry (at 15 min) and by confocal microscopy after 1-, 5-, 15-, and 30-min incubation. These results are shown in FIG. 2. The bar in FIG. 2 represents 5 μm. The S-palmitoyalted and N-palmitoyalted peptides were taken up much more rapidly and to much higher concentrations into macrophages than were non-palmitoylated peptides, as indicated by the stronger staining of the palmitoylated peptides—the palmitoylated peptides could easily be visualised inside the cells after 1 min, whereas the non-palmitoylated peptides could barely be visualised, even after 30 min incubation. This is also seen in the flow cytometric plot at 15 min, where the fluorescence intensity of staining with the palmitoylated peptides is several logs higher than the non-palmitoylated peptide.

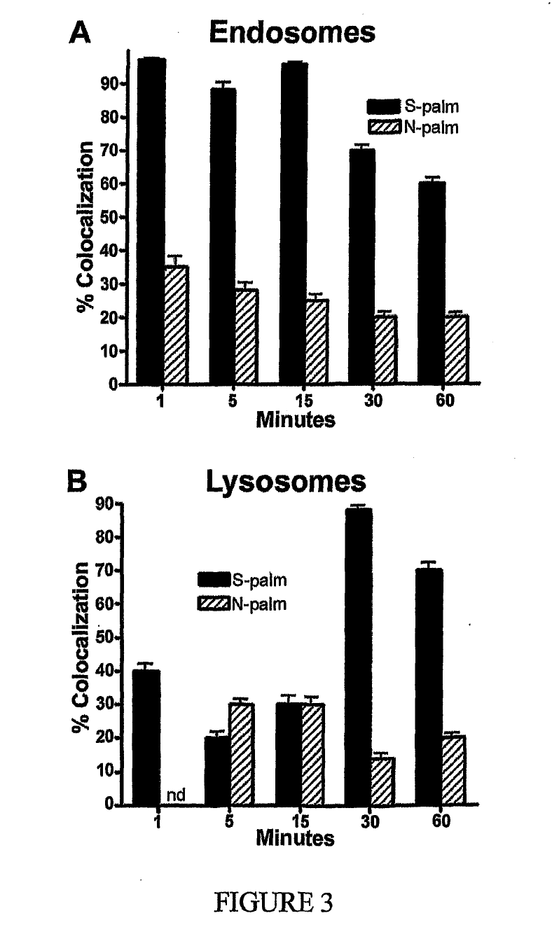

However, there were differences in the route of uptake of peptide as shown in FIG. 3. The percentage of endosomes or lysosomes that colocalised with peptide was determined using the colocalisation module of the Zeiss LSM Image Browser software. S-palm peptide colocalised rapidly and strongly with endosomes. N-palm peptide did not colocalise strongly with endosomes in most cells. Further, S-palm peptide colocalised strongly with lysosomes. N-palm peptide did not colocalise strongly with lysosomes in most cells, and there was no time-dependent increase of this localisation. The bars in the graphs in FIG. 3 represent the percentage colocalisation (mean±SE) in at least 100 cells. The term “nd” means not done.

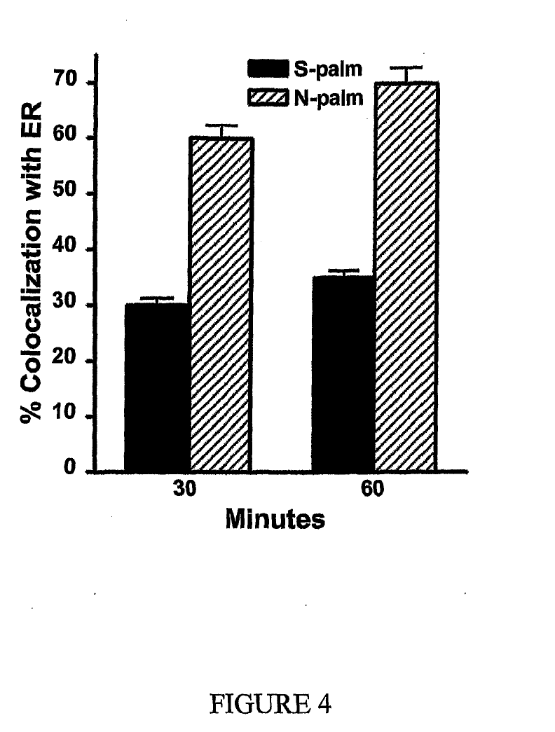

In contrast, N-palm peptide, but not S-palm peptide colocalised strongly with endoplasmic reticulum (ER) as shown in FIG. 4. The results show percentage colocalisation after incubation with biotinylated peptide for 30 or 60 minutes and staining to detect ER or peptide. Colocalisation of N-palm peptide and ER increased after 30 minutes of incubation, compared with colocalisation of S-palm peptide with ER.

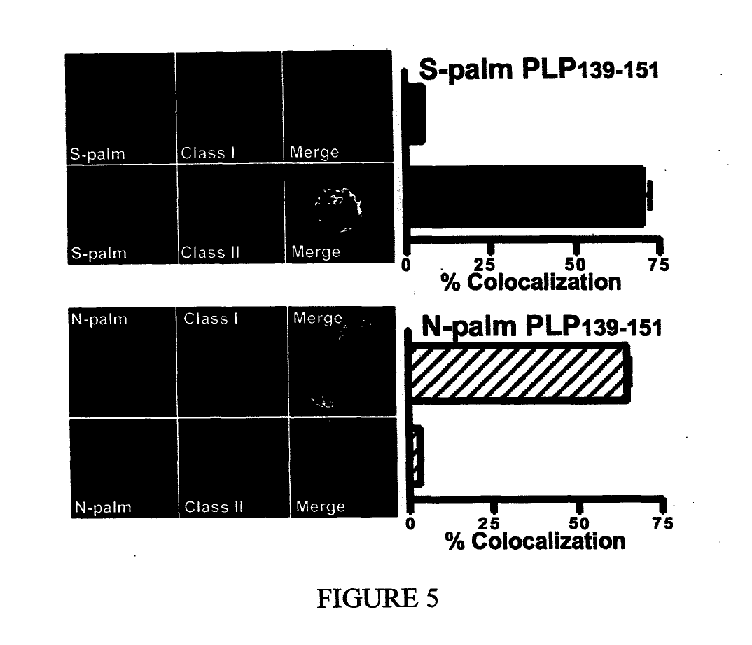

Further, the results also demonstrated that S-palm peptide colocalised strongly with MHC class II, but there was only a small percentage of S-palm that colocalised with MHC class I (p<0.0001). In contrast, N-palm peptide colocalised strongly with MHC class I and to a much lesser degree with MHC class II (p<0.0001). These results are shown in FIG. 5. Thus S-palm peptide was found to be presented by MHC class II molecules, whereas N-palm peptide was found to be presented by MHC class I molecules.

### Example 4

### Method

The following peptides produced in Example 1 were used:

| | | |
|---|---|---|
| native peptide | W144 | 2HN-HCLGKWLGHPDKF-COOH |
| | | (SEQ ID NO: 3) |
| APL | Q144 | 2HN-HCLGKQLGHPDKF-COOH |
| | | (SEQ ID NO: 4) |
| lipoAPL | S-palm | 2HN-HC(palm)LGKQLGHPDKF-COOH |
| | Q144 | (SEQ ID NO: 5) |

One mg per mL of W144 peptide, either alone or together with Q144 peptide or S-palm Q144 peptide at different molar ratios, was emulsified in an equal volume of complete Freund's adjuvant containing an additional 4 mg/ml Mycobacterium tuberculosis H37Ra. 200 μl of the emulsion (i.e. 100 μg W144) was then injected subcutaneously in a single site on the back of each SJL/J mouse. Each mouse also received 300 ng pertussis toxin intravenously on the same day as the peptide emulsion and then 3 days later.

The mice were inspected and weighed daily and scored for disease using a 5 point severity scale: 0=no disease; 1=decreased tail tone; 2=no tail tone; 3=hind limb weakness; 4=hind limb paralysis; 5=moribund.

Mice were followed for up to 40 days, provided they did not show signs of developing disease.

### Results

TABLE 5
Effect of different peptide emulsions on induction of EAE

| | Inci- | Mean day | |
|---|---|---|---|
| Immunisation (ratio) | dence | of onset | Mean severity* |
| W144 | 4/4 | 11.5 | 4.3 |
| W144/Q144 (1:6) | 3/4 | 11.3 | 2.8 |
| W144/S-palm Q144 (1:1) | 1/4 | 21 | 2 |
| W144/S-palm Q144 (1:6) | 0/4 | — | — |

*Mean maximum severity of the mice that developed EAE, based on a 5 point scale (0 = no EAE)

### Conclusions

Co-immunisation of S-palm Q144 at a ratio of 1:6 with the encephalitogenic peptide W144 protected the animals from EAE. By comparison, the non-palmitoylated APL Q144 at the same dose only reduced the severity of the disease.

Thus S-thiopalmitoylation of the APL enhanced the protective effect of the APL on EAE, and a lower dose was needed compared to the non-acylated APL to have the protective effect.

### Example 5

### Method

The following peptides produced in Example 1 were used:

| | | |
|---|---|---|
| native peptide | F188 | 2HN-NTWTTCQSIAFPSK-COOH |
| | | (SEQ ID NO: 6) |
| APL | A188 | 2HN-NTWTTCQSIAAPSK-COOH |
| | | (SEQ ID NO: 8) |
| lipo APL | S-palm | 2HN-NTWTTC(palm)QSIAAPS |
| | A188 | K-COOH |
| | | (SEQ ID NO: 9) |

One mg per mL of F188 peptide, either alone or together with A188 peptide or S-palm A188 peptide at different molar ratios, was emulsified in an equal volume of complete Freund's adjuvant containing an additional 4 mg/ml Mycobacterium tuberculosis H37Ra. 200 μl of the emulsion (i.e. 100 μg F188) was then injected subcutaneously in a single site on the back of each SJL/J mouse. Each mouse also received 300 ng pertussis toxin intravenously on the same day as the peptide emulsion and then 3 days later.

The mice were inspected and weighed daily and scored for disease using a 5 point severity scale: 0=no disease; 1=decreased tail tone; 2=no tail tone; 3=hind limb weakness; 4=hind limb paralysis; 5=moribund.

Mice were followed for up to 40 days, provided they did not show signs of developing disease.

### Results

TABLE 6
Effect of different peptide emulsions on induction of EAE

| | Inci- | Mean day | |
|---|---|---|---|
| Immunisation (ratio) | dence | of onset | Mean severity |
| F188 | 8/8 | 11.3 ± 0.5 | 2.8 ± 0.3 |
| F188/A188 (1/5) | 2/4 | 13.5 | 0.75* |
| F188/A188 (1/1) | 4/8 | 11.5 ± 0.5 | 1.4 ± 0.6 |
| F188/A188 (1/0.2) | 3/4 | 11.6 | 2 |
| F188/A188 (1/0.1) | 4/4 | 12.25 | 2.5 |
| F188/S-palm A188 (1/1) | 1/8 | 14 | 0.1 ± 0.1*** |
| F188/S-palm A188 (1/0.2) | 3/7 | 14.7 ± 1.8 | 0.5 ± 0.3** |
| F188/S-palm A188 (1/0.1) | 3/8 | 11.6 ± 0.3 | 0.9 ± 0.5* |

*p < 0.05;
**p < 0.01;
***p < 0.002 compared to F188-immunised mice

### Conclusions

The APL A188 was protective when injected at a dose 5 times greater than the encephalitogenic peptide F188.

The S-palm APL A188 was protective when injected at a dose 5 times lower than the encephalitogenic peptide F188.

Thus S-thiopalmitoylation of the APL enhanced the protective effect of the APL on EAE, and a lower dose was needed compared to the non-acylated APL to have the protective effect.

### Example 6

### Method

The following peptides produced in Example 1 were used:

| | | |
|---|---|---|
| native peptide | F188 | 2HN-NTWTTCQSIAFPSK-COOH |
| | | (SEQ ID NO: 6) |
| APL | A188 | 2HN-NTWTTCQSIAAPSK-COOH |
| | | (SEQ ID NO: 8) |
| lipo APL | S-palm | 2HN-NTWTTC(palm)QSIAAPS |
| | A188 | K-COOH |
| | | (SEQ ID NO: 9) |

A 100 nM solution of F188 peptide was emulsified in an equal volume of complete Freund's adjuvant containing an additional 4 mg/ml Mycobacterium tuberculosis H37Ra. 200 μl of the emulsion (i.e. 50 nM F188) was then injected subcutaneously in a single site on the back of each SJL/J mouse. Each mouse also received 300 ng pertussis toxin intravenously on the same day as the peptide emulsion and then 3 days later.

On day 10 after injection of F188, just prior to the onset of clinical signs of disease in the mice, the mice were injected with either A188 or S-palm A188 in PBS subcutaneously, either at 50 nM/mouse (1:1 F188:APL or S-palm APL) or 10 nM/mouse (1:0.2 F188:APL or S-palm APL).

The mice were inspected and weighed daily and scored for disease using a 5 point severity scale: 0=no disease; 1=decreased tail tone; 2=no tail tone; 3=hind limb weakness; 4=hind limb paralysis; 5=moribund.

Mice were followed for up to 40 days, provided they did not show signs of developing disease.

### Results

TABLE 7
Effect of different peptide emulsions on induction of EAE

| | Inci- | Mean day | |
|---|---|---|---|
| APL (ratio) | dence | of onset* | Mean severity |
| None | 10/11 | 11.8 ± 0.4 | 2.8 ± 0.3 |
| A188 (1:1) | 9/12 | 14.9 ± 1.2 | 1.3 ± 0.4 |
| A188 (1:0.2) | 4/4 | 11.5 ± 0.5 | 3.3 ± 0.4 |
| S-palm A188 (1:1) | 5/12** | 13.2 ± 0.7 | 1.0 ± 0.4** |
| S-palm A188 (1:0.2) | 6/9 | 13.8 ± 2.0 | 1.8 ± 0.6 |

*mean day of onset of mice that developed EAE
**p < 0.05 compared to controls (no APL)

### Conclusions

The 1:1 A188 immunisation protocol, but not the 1:0.2 A188 immunisation protocol, had some effects on induction of EAE. In contrast, both doses of S-palm-A188 reduced the incidence of disease, the day of onset of EAE and the severity of EAE in the mice.

### Example 7

### Method

The following peptides produced in Example 1 were used:

| | | |
|---|---|---|
| lipo | S-palm- | 2HN-NTWTTC(palm)QSIAFPSK-COOH |
| native | PLP178-191 | (SEQ ID NO: 7) |
| peptide | | |
| APL | A188 | 2HN-NTWTTCQSIAAPSK-COOH |
| | | (SEQ ID NO: 8) |
| lipo APL | S-palm A188 | 2HN-NTWTTC(palm)QSIAAPSK-COOH |
| | | (SEQ ID NO: 9) |

SJL/J mice were injected subcutaneously with 100 μg S-palm-F188 (S-palm-PLP178-191), i.e. 100 μl of a 1 mg/ml solution emulsified in an equal volume (i.e. 100 μl Complete Freund's Adjuvant containing an extra 4 mg/mL Mycobacterium tuberculosis H37Ra. Each mouse also received 300 ng pertussis toxin intravenously on the same day as the peptide emulsion and then 3 days later.

The mice were then inspected and scored for disease using a 5 point severity scale: 0=no disease; 1=decreased tail tone; 2=no tail tone; 3=hind limb weakness; 4=hind limb paralysis; 5=moribund.

When the mice reached a score between 2.0 or 2.5, each mouse was injected subcutaneously with 100 μL PBS (n=6), 100 μL of 1 mg/mL A188 in PBS (n=6), or 100 μL of 1 mg/mL S-palm-A188 in PBS (n=7).

Mice were then followed for 7 days, and scored each day.

### Results

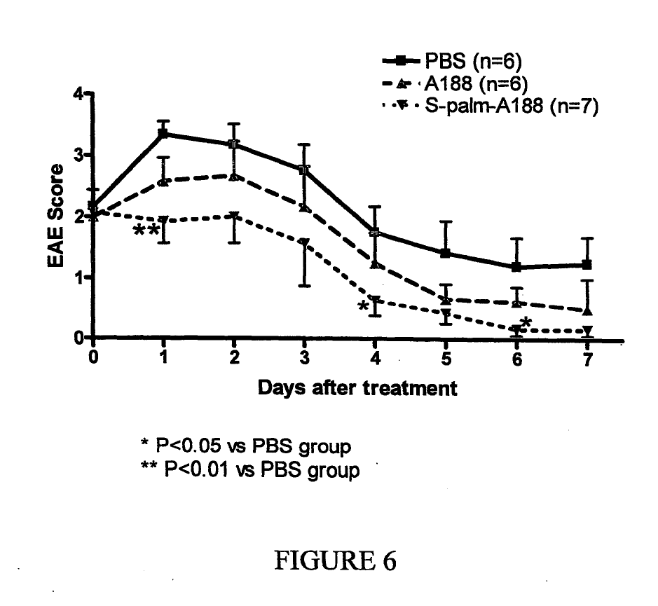

The results are demonstrated in FIG. 6, where it can be seen that the control (PBS) group continued to develop more severe EAE, whereas the mean score of the S-palm-A188-treated group did not increase above the score that the mice were at when they were injected with the treatment. The response of the mice treated with A188 was intermediate between the control group and the S-palm-A188-treated group.

### Conclusions

The results from this example indicate that the S-palm-A188 will be effective in a therapeutic situation.

### Example 8

### Method

The following peptides produced in Example 1 were used:

| | | |
|---|---|---|
| native peptide | F188 | 2HN-NTWTTCQSIAFPSK-COOH |
| | | (SEQ ID NO: 6) |
| APL | A188 | 2HN-NTWTTCQSIAAPSK-COOH |
| | | (SEQ ID NO: 8) |
| lipo APL | S-palm | 2HN-NTWTTC(palm)QSIAAPS |
| | A188 | K-COOH |
| | | (SEQ ID NO: 9) |

Mice were immunised subcutaneously with 100 μl of a 1 mg/ml solution of either A188 or S-palm A188 emulsified in an equal volume of complete Freund's adjuvant. Lymph nodes were removed 10 days after injection of the peptides and single cell suspensions of lymph node cells (LNC) were prepared.

For RNA analysis, the LNC were stimulated for 4 hours in vitro in the presence of no antigen, or 20 μg/ml of A188 or the native peptide PLP178-191. After that time, cells were harvested and frozen for later RNA extraction. For analysis of CD4+CD25+ T regulatory cells, LNC were stimulated for 3 days in vitro in the presence of no antigen, or 20 μg/ml of A188 or the native peptide PLP178-191. They were then harvested and stained with antibodies for CD4 and CD25 prior to flow cytometric analysis.

RNA extracted from LNC was then analysed by RT-PCR. Flow cytometric analysis of CD4+CD25+T regulatory cells was also carried out on the LNC.

### Results

Analysis by RT-PCR of the extracted RNA showed significant increases in expression of the Th2-related genes IL-10, IL-13 and GATA-3 in the LNC from mice immunized with S-palm A188, in comparison to control mice and those immunised with A188, upon stimulation with either the APL or the native peptide (PLP178-191).

In addition, the S-palm A188-immunised mice significantly upregulated the transcription factor FoxP3, which is a marker of regulatory T cells, upon stimulation with either APL or native peptide.

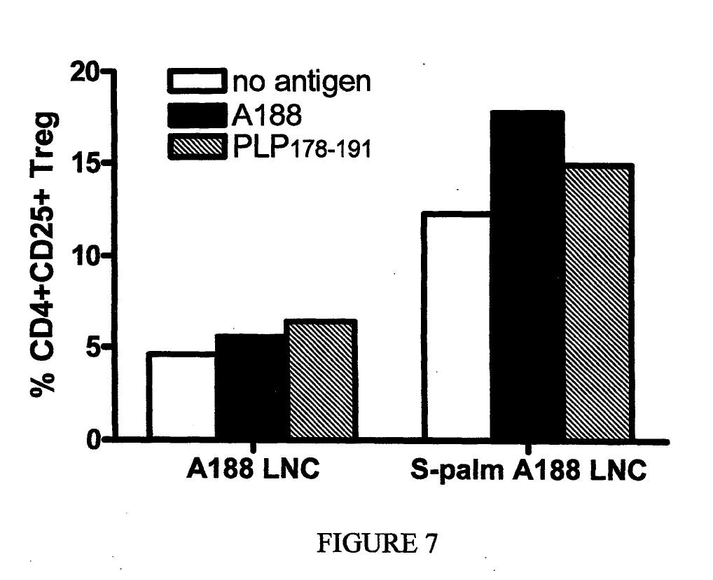

The flow cytometric analysis of the LNC from mice immunised with either A188 or S-palm A188, confirmed that the S-palm A188 induces an increase in the number of regulatory cells. These results are shown in FIG. 7. FIG. 7 shows the percentages of regulatory T cells in LNC from mice immunized with either A188 or S-palm A188. The baseline level of regulatory T cells (i.e. no antigen group) is increased in the S-palm A188-treated mice, and those levels are increased further upon stimulation of the LNC with either A188 or PLP178-191. These results may explain in part the increased immunomodulatory capacity of the S-palm A188.

### Example 9

Example 9 describes a proposed experiment used to test the ability of an APL and a thiopalmitoylated form of the APL for preventing or treating type II collagen-induced arthritis using the mouse model described in Coutenay et al.

### Method

The following peptides are to be used:

| | | |
|---|---|---|
| native | Cys CII256-271 | CGKPGIAGFKGEQGPKG |
| peptide | | (SEQ ID NO: 22) |
| APL | Cys | CGKPGIAAFKGEQGPKG |
| | CII256-271 | (SEQ ID NO: 23) |
| | A262 | |
| S-palm APL | Cys(palm) | C(palm)GKPGIAAFKGEQGPKG |
| | CII256-271 | (SEQ ID NO: 24) |
| | A262 | |

The native peptide and the APL are described in Wakamatsu et al.

To induce the arthritis, DBA/1 mice will be immunised intradermally with 100 μg bovine type II collagen (CII) in Complete Freund's adjuvant. Each mouse will also receive a booster dose on day 21 by intraperitoneal injection of 100 μg of CII.

Mice will be treated with three injections each of 333 μg of APL intraperitoneally (total 1 mg) on days 24, 26, and 28 after the first immunisation with CII, using the same protocol as described in Wakamatsu et al. In Wakamatsu et al., the mice were shown to develop the disease 21-25 days (typically around day 24) after the initial immunisation of CII. Control mice will receive the injection vehicle according to the same schedule. S-palm APL will be tested in at the same concentration, and also at 100, 33 and 10 μg per injection, to determine whether lower doses of S-palm APL are more effective than the APL.

The animals will be observed at daily intervals and evaluated for the severity or arthritis by scoring each paw. The score will range from 0 to 3 (0, no swelling or redness; 1, swelling or redness in one joint; 2, involvement of two or more joints; 3, severe arthritis of the entire paw and joints). The total score of each animal will be the sum score of all four paws.

### Example 10

Example 10 describes a proposed experiment used to test the efficacy of an APL and a thiopalmitoylated form of the APL for preventing or treating type I diabetes in NOD mice.

### Method

The following peptides are to be used:

| | | |
|---|---|---|
| native | insulin B₉₋₂₃ | SHLVEALYLVCGERG |
| peptide | | (SEQ ID NO: 25) |
| APL | insulin | SHLVEALALVCGERG |
| | B₉₋₂₃ A16 | (SEQ ID NO: 26) |
| S-palm APL | insulin | SHLVEALALVC(palm)GERG |
| | B₉₋₂₃Cys(palm) | (SEQ ID NO: 27) |
| | A16 | |

The native peptide and the APL are described in Alleva et al.

Female NOD mice will be used in these experiments. These mice provide a spontaneous model of autoimmunity, and typically develop frank diabetes between 15-20 weeks of age (Alleva et al., and Cameron et al.).

To test the preventative effects of the APL and S-palm APL, prediabetic female NOD mice (i.e., 4-week-old, insulitis-free) will be treated with weekly subcutaneous injections of 400 μg APL for 12 weeks and then one injection every 2 weeks until mice reach 39 weeks of age. Control mice will receive the injection vehicle according to the same schedule. S-palm APL will be tested at the same concentration, and also at 100, 40 and 10 μg per injection, to determine whether lower doses of S-palm APL are more effective than the APL.

To test the therapeutic effects of the APL and S-palm APL, 12- to 20-week-old female NOD mice with insulitis will receive a single subcutaneous injection of 100 μg vehicle, APL, or S-palm APL, emulsified in incomplete Freund's adjuvant (IFA).

Blood glucose will be monitored using a glucometer (Encore Glucometer; Bayer) at weekly intervals, beginning at 10 weeks of age. Mice with blood glucose levels equal to or greater than 200 mg/dl on two consecutive occasions will be considered diabetic.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

### BIBLIOGRAPHY

1. Alleva D et al., 2002, Diabetes, 51:2126-2134.
2. Anton et al., 1998, Scand J Immunol, 47:116-121.
3. Araki et al., 2001, Br J Haematol, 114:681-689.
4. Bai et al., 2002, Int J Oncol, 20:247-53.
5. Battye and Shortman, 1991, Curr. Opin. Immunol., 3:238-241.
6. Bielekova et al., 2000, Nat Med, 6:1167-1175.
7. Boots A, et al., 2007, Arthritis Res Ther, 9(4):R71.
8. Cameron M J et al., 1998, Diabetes Metab. Rev., 14:177-185.
9. Chen et al., 2001, Clin Immunol, 98:280-292.
10. Chen T. et al., 2004, J Clin Invest, 113:1754-1762.
11. Clark, 2002, J Leukoc Biot, 71:388-400.
12. Coutenay, J S et al., 1980, Nature, 283:666-668.
13. Crowe et al., Ann Neurol, 2000, 48:758-765.
14. Davis et al., 1994, J Immunol Meth, 175:247-257.
15. Ely et al., 2005, J Immunol, 2005, 174:5593-5601.
16. Erickson-Miller et al., 1990, Int J Cell Cloning, 8:346-356.
17. Farkas et al., 2001, Am J Pathol, 159:237-243.
18. Fearnley et al., 1999, Blood, 93:728-736.
19. Fleming et al., 2001, Exp Hematol, 29:943-951.
20. Freudenthal and Steinman, 1990, Proc Natl Acad Sci USA, 87:7698-7702.
21. Garderet et al., 2001, J Hematohther Stem Cell Res, 10:553-567.
22. Gebe J et al., 2004, Eur J Immunol, 34:3337-3345.
23. Giessmann et al. 1998, J Exp Med, 187:961-966.
24. Greer et al., 1996, J Immunol, 156:371-379.
25. Greer et al., 1997, Neurochem Res, 22(4):541-547.
26. Greer et al., 2001, J Immunol, 166:6907-6913.
27. Groopman et al., 1987, N Engl J Med, 317:593-598.
28. Grouard et al., 1997, J Exp Med, 185:1101-1111.
29. den Haan et al., 1 Feb. 2002, Blood, 99(3):985-992.
30. Hacker et al., 2002, Immunology, 105:245-251.
31. Hart and Fabre, 1981, J Exp Med, 154(2):347-361.
32. Hart and McKenzie, 1988, J Exp Med, 168(1):157-170.
33. Hartemann-Heurtier et al., 2004, J Immunol, 172:915-922.
34. Hill et al., 1995, J Leukoc Biot, 58:634-642.
35. Hofstetter et al., 2007, J Immunol, 178:1372-1378.
36. del Hoyo et al., 2002, Nature, 415:1043-1047.
37. Kaisho and Akira, 2002, Biochem Biophys Acta, 1589:1-13.
38. Kappos et al., 2000, Nat Med, 6:1176-1182.
39. Karin et al., 1994, J Exp Med, 180:2227-2237.
40. Katz-Levy et al., 1997, Proc Natl Acad Sci USA, 94:3200-3205.
41. Kelly et al., 2001, Cell Mol Biol (Noisy-le-grand), 47:43-54.
42. Kinnunen T. et al., 2007, J Allergy Clin Immunol, 119: 965-72.
43. Koski et al., 2001, Crit. Rev Immunol, 21:179-189.
44. Kuchroo et al., 1994, J. Immunol, 173(7):3326-36.
45. Li R et al., 2006, Clin Immunol, 118:317-323.
46. Loudovaris et al., 2001, J Hematother Stem Cell Res, 10:569-578.
47. Lu et al., 1994, J Exp Med, 179:1823-1834.
48. Mackensen et al., 2000, Int J Cancer, 86:385-392.
49. de Marquesini et al., 2008, Eur. J. Immunol, 38:240-249.
50. McIlroy et al., 2001, Blood, 97:3470-3477.
51. Metcalf and Burgess, 1982, J Cell Physiol, 111:275-283.
52. Myers L et al., 1993, J Immunol, 150:4652-4658.
53. Naito Y et al., 2006, Ann Rheum Dis, 65:269-271.
54. Nestle et al., 1998, Nat Med, 4:328-332.
55. Nicholson et al., 1995, Immunity, 3:397-405.
56. O'Doherty et al., 1993, J Exp Med, 178:1067-1078.
57. O'Doherty et al., 1994, Immunology, 82:487-493.
58. O'Sullivan and Thomas, 2002, J Immunol, 168:5491-5498.
59. Oehler et al., 1998, J Exp Med, 187:1019-1028.
60. Oehler et al., 2000, Ann Hematol, 79:355-362.
61. Offenhäusser M et al., 2002, J Neuroimmunol, 129:97-105.
62. Ohnishi Yi et al., 2006, Mod Rheumatol, 16:226-228.
63. Pavli et al., 1990, Immunology, 70(1):40-47.
64. Prakken B et al., 2002, Arthritis and Rheumatism, 46(7):1937-1946.
65. Pulendran et al., 2000, J Immunol, 165:566-572.
66. Rissoan et al., 1999, Science, 283:1183-1186.
67. Robinson et al., 2000, J Hematother Stem Cell Res, 9:711-720.
68. Romani et al., 1994, J Exp Med, 180:83-93.
69. Romani et al., 1996, J Immunol Meth, 196:137-151.
70. Santiago-Scharz et al., 2001, J Immunol, 167:1758-1768.
71. Shen Z et al., 2006, J Am Acad Dermatol, 54:992-1002.
72. Sorg et al., 2001, Exp Hematol, 29:1289-1294.
73. Stewart et al., 2008, Org. Biomol. Chem., 6:2242-2255.
74. Stobl et al., 1996, J Immunol, 157:1499-1507.
75. Strobl et al., 1997a, Blood, 90:1425-1434.
76. Strobl et al., 1997b, dv Exp Med Biot, 417:161-165.
77. Thomas and Lipsky, 1994, J Immunol, 153:4016-4028.
78. Thomas et al., 1993, J Immunol, 151(12):6840-6852.
79. Thomas et al., 1994a, J Immunol, 153:4016-4028.
80. Thomas et al., 1994b, Arthritis Rheum, 37(3):445-6.
81. Thurner et al., 1999, J Immunol Methods, 223:1-15.
82. Vremec and Shortman, 1997, J Immunol, 159(2):565-573.
83. Vremec et al., 2000, J Immunol, 164(6):2978-86.
84. J. Immunol. 2000 Mar. 15; 164(6):2978-86.
85. Vuckovic et al., 1998, Exp Hematol, 26:1255-1264.
86. Wakamatsu et al., 2009, Mod Rheumatol, 19: 366-371.
87. Zheng et al., 2000, J Hematother Stem Cell Res, 9:453-464.

1. A peptide conjugate for suppressing or otherwise inhibiting an MHC class II response, the peptide conjugate comprising a peptide comprising an amino acid sequence that suppresses or otherwise inhibits an unwanted or undesirable immune response and that is processed and presented by an MHC class II molecule, and a lipid moiety which is attached to the peptide through a thioester linkage, or a pharmaceutically acceptable salt thereof.

2. (canceled)

3. The peptide conjugate of claim 1, wherein the peptide comprises an altered peptide ligand.

4. The peptide conjugate of claim 1, wherein the peptide sequence comprises 5-40 amino acid residues.

5. The peptide conjugate of claim 4, wherein the peptide sequence comprises 10-20 amino acid residues.

6. The peptide conjugate of claim 1, wherein the peptide includes at least one cysteine residue.

7. The peptide conjugate of claim 6, wherein the thioester linkage is through the thiol group of the cysteine residue.

8. The peptide conjugate of claim 1, wherein the lipid moiety comprises a fatty acid having 8-18 carbon atoms.

9. The peptide conjugate of claim 8, wherein the fatty acid is tetradecanoic acid or hexadecanoic acid.

10. A composition comprising the peptide conjugate of claim 1 and a pharmaceutically acceptable excipient or diluent or adjuvant.

11. A composition consisting essentially of the peptide conjugate of claim 1.

12. A composition consisting of the peptide conjugate of claim 1.

13. A composition comprising the peptide conjugate of claim 1 but excluding a separate antigen that corresponds to the sequence of the peptide, which antigen elicits the unwanted or undesirable immune response.

14. A method of making the peptide conjugate of claim 1, the method comprising linking a peptide comprising an amino acid sequence that suppresses or otherwise inhibits an unwanted or undesirable immune response and that is processed and presented by an MHC class II molecule to a lipid moiety through a thioester linkage.

15. (canceled)

16. A method for suppressing or otherwise inhibiting an unwanted or undesired immune response in a subject, the method comprising administering to the subject the peptide conjugate of claim 1.

17. A method for suppressing or otherwise inhibiting an unwanted or undesired immune response to a target antigen in a subject, the method comprising administering to the subject the peptide conjugate of claim 1, wherein the sequence of the peptide corresponds to the sequence of the target antigen.

18. A method for preventing, inhibiting, treating or decreasing an autoimmune, an allergic immune or an allograft immune response in a subject, the method comprising administering to the subject the peptide conjugate of claim 1.

19. (canceled)

20. A method for preventing, inhibiting, treating or decreasing an autoimmune, an allergic immune or an allograft immune response to a target antigen in a subject, the method comprising administering to the subject the peptide conjugate of claim 1, wherein the sequence of the peptide corresponds to the sequence of the target antigen.

21-30. (canceled)

31. A method for producing an immunosuppressive antigen presenting cell, the method comprising contacting an antigen presenting cell or antigen presenting cell precursor with the peptide conjugate of claim 1 for a time and under conditions sufficient for the peptide or a processed form thereof to be presented by the antigen presenting cell or antigen presenting cell precursor.


SEQUENCE LISTING (27 sequences)

SEQ ID NO: 1 - protein, 13 aa, Artificial Sequence
  A synthetic peptide
HCLGKWLGHPDKF

SEQ ID NO: 2 - protein, 13 aa, Artificial Sequence
  A synthetic peptide
HCLGKWLGHPDKF

SEQ ID NO: 3 - protein, 13 aa, Artificial Sequence
  A synthetic peptide
HCLGKWLGHPDKF

SEQ ID NO: 4 - protein, 13 aa, Artificial Sequence
  A synthetic peptide
HCLGKQLGHPDKF

SEQ ID NO: 5 - protein, 13 aa, Artificial Sequence
  A synthetic peptide
HCLGKQLGHPDKF

SEQ ID NO: 6 - protein, 14 aa, Artificial Sequence
  A synthetic peptide
NTWTTCQSIAFPSK

SEQ ID NO: 7 - protein, 14 aa, Artificial Sequence
  A synthetic peptide
NTWTTCQSIAFPSK

SEQ ID NO: 8 - protein, 14 aa, Artificial Sequence
  A synthetic peptide
NTWTTCQSIAAPSK

SEQ ID NO: 9 - protein, 14 aa, Artificial Sequence
  A synthetic peptide
NTWTTCQSIAAPSK

SEQ ID NO: 10 - protein, 14 aa, Artificial Sequence
  A synthetic peptide
KTTICGKGLSATVT

SEQ ID NO: 11 - protein, 14 aa, Artificial Sequence
  A synthetic peptide
KTTICGKGLSATVT

SEQ ID NO: 12 - protein, 14 aa, Artificial Sequence
  A synthetic peptide
KTTICGKGLSATVT

SEQ ID NO: 13 - protein, 9 aa, Homo sapiens
FLRGRAYGL

SEQ ID NO: 14 - protein, 9 aa, Homo sapiens
VLHDDLLEA

SEQ ID NO: 15 - protein, 9 aa, Mus musculus
LYLVCGERG

SEQ ID NO: 16 - protein, 10 aa, Homo sapiens
FKGEQGPKGE

SEQ ID NO: 17 - protein, 10 aa, Homo sapiens
FKGEAGPAGE

SEQ ID NO: 18 - protein, 10 aa, Homo sapiens
FKAEAGPAGE

SEQ ID NO: 19 - protein, 10 aa, Homo sapiens
FKGEAGPAAE

SEQ ID NO: 20 - protein, 15 aa, Homo sapiens
VPPGECFIQFLSEPT

SEQ ID NO: 21 - protein, 17 aa, Homo sapiens
AKPVVHLFANIVTPRTP

SEQ ID NO: 22 - protein, 17 aa, Artificial Sequence
  A synthetic peptide
CGKPGIAGFKGEQGPKG

SEQ ID NO: 23 - protein, 17 aa, Artificial Sequence
  A synthetic peptide
CGKPGIAAFKGEQGPKG

SEQ ID NO: 24 - protein, 17 aa, Artificial Sequence
  A synthetic peptide
CGKPGIAAFKGEQGPKG

SEQ ID NO: 25 - protein, 15 aa, Artificial Sequence
  A synthetic peptide
SHLVEALYLVCGERG

SEQ ID NO: 26 - protein, 15 aa, Artificial Sequence
  A synthetic peptide
SHLVEALALVCGERG

SEQ ID NO: 27 - protein, 15 aa, Artificial Sequence
  A synthetic peptide
SHLVEALALVCGERG